(19) United States
(12) Patent Application Publication
(10) Pub. No.: US 2025/0253046 A1
(43) Pub. Date: Aug. 7, 2025

(54) MASS SPECTROMETRY METHODS FOR DETERMINING GLYCOPROTEOFORM-BASED BIOMARKERS

(71) Applicant: Northwestern University, Evanston, IL (US)

(72) Inventors: Stephen Matthew Patrie, Evanston, IL (US); Jiana Duan, Evanston, IL (US)

(21) Appl. No.: 18/855,193

(22) PCT Filed: Apr. 10, 2023
(86) PCT No.: PCT/US2023/065590

§ 371 Date: Oct. 8, 2024

Related U.S. Application Data
(60) Provisional application No. 63/362,708, filed on Apr. 8, 2022.

(51) Int. Cl. G16H 50/20; G01N 33/68; G06N 20/20; G16B 40/10; G16B 45/00

(52) U.S. Cl. CPC G16H 50/20; G01N 33/6848; G06N 20/20; G16B 40/10; G16B 45/00; G01N 2333/4728; G01N 2800/26; G01N 2800/56

(57) ABSTRACT

Disclosed herein are mass spectrometry methods for determining glycoproteoform-based biomarkers. The methods comprise identifying, with a processor from mass spectrometry data of the glycoprotein, a set of glycoproteoforms where each of the glycoproteoforms have a measurable intact mass; generating, with the processor from the identified set of glycoproteoforms, a glycoproteoform network separated by saccharide features, determining, with the processor from the glycoproteoform network, a site-independent prediction of N-glycans mapped to biosynthesis pathways; and generating, with the processor from the determined N-glycans mapped to biosynthesis pathways, a glycan structure. The methods may be used to analyze a glycoprotein in a subject, analyze disease progression, or identify disease onset or a recovery.

### CROSS-REFERENCE TO RELATED APPLICATIONS

The present application claims priority to U.S. Provisional Patent Application No. 63/362,708, filed Apr. 8, 2022, the entire contents of which are hereby incorporated by reference.

### STATEMENT REGARDING FEDERALLY SPONSORED RESEARCH

This invention was made with government support under 5R01GM115739-06 awarded by National Institutes of Health. The government has certain rights in the invention.

### BACKGROUND OF THE INVENTION

Glycosylation occurs on approximately half of all proteins, serving to modulate protein interactions, drug (biotherapeutics or biosimilars) activity and stability, or serve as biomarkers for diseases such as cancer and neurodegenerative diseases. As a result, there is a need for methods for determining protein glycosylation and glycoproteofrom-based biomarkers.

### BRIEF SUMMARY OF THE INVENTION

Disclosed herein are mass spectrometry methods for determining glycoproteoform-based biomarkers from intact data. The methods comprise identifying, with a processor from mass spectrometry data of the glycoprotein (G), a set of glycoproteoforms (gp∈G) where each of the glycoproteoforms (gpᵢ) have a measurable intact mass; generating, with the processor from the identified set of glycoproteoforms (gp∈G), a glycoproteoform network separated by saccharide features, determining, with the processor from the glycoproteoform network, a site-independent prediction of N-glycans mapped to biosynthesis pathways; and generating, with the processor from the determined N-glycans mapped to biosynthesis pathways, a glycan structure.

One aspect of the technology provides for a method for analyzing a glycoprotein (G) in a subject. The method comprises obtaining a biospecimen from the subject; analyzing, with a mass spectrometer, the biospecimen to generated mass spectrometry data of the glycoprotein; identifying, with a processor from the mass spectrometry data of the glycoprotein, a set of glycoproteoforms (gp∈G) where each of the glycoproteoforms (gpᵢ) have a measurable intact mass; generating, with the processor from the identified set of glycoproteoforms (gp∈G), a glycoproteoform network separated by saccharide features, determining, with the processor from the glycoproteoform network, a site-independent prediction of N-glycans mapped to biosynthesis pathways; and generating, with the processor from the determined N-glycans mapped to biosynthesis pathways, a glycan structure. In some embodiments, the method further comprises determining disease onset or disease recovery for the subject from the glycoproteoform network separated by saccharide features, the site-independent prediction of N-glycans mapped to biosynthesis pathways, the glycan topology, or any combination thereof. In some embodiments, the method further comprises administering a treatment to the subject in need of a treatment for disease onset.

Another aspect of the technology provides for a method for analyzing disease progression in a subject. The method comprises obtaining two or more biospecimens from the subject at different time points; generating a glycoproteoform network or a glycan structure according to the methods described herein for the two or more obtained biospecimens; and identifying an indicia of disease onset or an indicia of disease recovery in the generated glycoproteoform network or the glycan structure between the two or more obtained biospecimens or a control. In some embodiments, the subject is diagnosed with sepsis or is suspected of developing, having, or having had sepsis.

Another aspect of the technology provides for identifying disease onset or recovery in a subject. The method comprises generating a glycoproteoform network or a glycan structure for the subject according to the methods according to the methods described herein; and identifying an indicia of disease onset or an indicia of disease recovery in the generated glycoproteoform network or the glycan structure for the subject in comparison to one or more of pre-disease onset subjects, diseased subjects, or recovered subjects. In some embodiments, the subject is diagnosed with sepsis or is suspected of developing, having, or having had sepsis.

These and other aspects of the invention will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Panel (A) shows a rendering highlights the branch progression for 335 common simple, hybrid and complex N-glycans with up to tetra-antennary branches that contain up to four sialic acid residues (this network was generated using Matlab Software, GlycoVis). Panel (B) shows that, of the 335 N-glycans, there are 70 unique Fₙ:Hₙ:GNₙ:SAₙ compositional isomers, each with a unique mass. For example, these 6 glycans each represent an isomer with the same sugar composition and mass. Panel (C) show that, for glycoproteins harboring up to 4 N-glycans, the model enumerates glycoproteoforms (gps) with each represented by a generic Fₙ:Hₙ:GNₙ:SAₙ glycan.

FIG. 2 shows the proteoform network analysis (PNA) workflow utilizes intact glycoproteoform data to: Panel (a) visualizes the gpᵢ∈G network connectivity where the directional flow of the network edges most often reflects the stepwise addition of monosaccharides (+F, +H, +GN, or +S) which directly correlate with the overlapping spectral shifts in the mass spectrum; Panel (b) show execution of a site-independent prediction of the type and intensity of standard N-glycans (highlighted) followed by predicted N-glycan pathway optimization to provide likely structural topologies (i.e., degree of branching, bisection status); Panel (c) shows generation of a range of network metadata used to describe and quantify network or subnetwork attributes and glycan moiety distributions; panel (d) compares two or more datasets at the intact or N-glycan level.

FIG. 3 is a graphical illustration of an example method for determining a topology of a molecule in accordance with one aspect of the present disclosure.

FIG. 4 is a block diagram illustrating an example of a computer system that can implement some aspects of the present disclosure.

FIG. 5: Panel (a) shows the F_{2,3}∈LPGDS network (see FIG. 14 for full map). The inset highlights the -:H:GN:S connectivity within the F₂ subset. Panel (b) shows a focused view on the F₂∈LPGDS network. The weighted means -:H:GN.- and standard deviation (beneath) for the individual S₀₋₄∈F₂ subsets. Monosaccharide-level heatmaps highlight the summed spectral intensity across the entire F₂∈LPGDS network (top) and it's S₀₋₄ subsets (bottom). A plot compares weighted mean H and GN with respect to S (b, lower right). Panel (c) shows the GN₈₋₁₁∈F₂ biograph with observed starting gp shown (node hue proportional to gpᵢ intensity). Panel (d) shows (left) Intensity-dependent activity of GalT and SiaT for GN₈₋₁₀. Relationship between predicted N-glycan structures indicate completeness of GalT and SiaT activity for the GN₈₋₁₀ subsets.

FIG. 6: Panel (a) shows L-PGDS glycopeptide data and their site-specific compositional information (right). Panel (b) shows a Venn diagram highlighting 14 predicted N-glycans overlapping between predicted and observed results. Bar graph shows comparison of each individual N-glycan (predicted vs observed, r=0.75) and their relative abundances. Putative O-glycan contributions that affect in silico predictions are highlighted in green (represented by 14** in the Venn diagram). Panel (c) shows a theoretical mass spectrum of all L-PGDS gps—approximated via intensity-weighted permutation of previously characterized CSF L-PGDS N-glycopeptides—compared to experimental top-down intact glycoprotein MS (r=0.26). The Venn diagram highlights 20 unique gps that are only observed in TDMS are due to O-glycosylation contributions and have minor abundances (0.2-5% RI). Panel (d) shows a comparison of the predicted and observed “brain-type” N-glycan biosynthetic pathways.

FIG. 7 provides mass spectrum and corresponding network plots for CSF L-PGDS (gp_{N-MAX}=2:10:10:4) in panel (a), EPO-236 (gp_{N-MAX}=3:15:12:6) in panel (b), and EPO-113 (gp_{N-MAX}=3:21:18:12) in panel (c). Green nodes and edges (with red labels) are indicators of a gp that exceed the gp_{N-MAX}. For EPO-113 the prediction of the number of LacNAc residues is possible using the combination of standard (blue) and LacNAc gp network plots (red) while still being able to readily identify O-glycosylation series.

FIG. 8: Panel (a) shows a comparison of EPO 112 (navy) and EPO 113 (red) mass spectrum. EPO 112.b (green) denotes a sub-series of gp that are unique to only EPO 112, whereas EPO 112.a is a series of shared gp with EPO 113. Panel (b) shows differential expression of each gp in EPO 112.a and EPO 113. The unique subset from EPO 112.b (green) is overlayed. Panel (c) shows differences in gp expression between two samples plotted against their intensity ratios. Data points highlighted in orange are unique to either EPO 112 (above 1 on the y-axis) and EPO 113 (below 1 on the y-axis). Data points in grey above or below 0 on the y-axis indicate greater abundance in one particular EPO dataset. Panel (d) shows average distributions of Hand GN vs S. EPO 113 is the only cell line that has complete sialation (12 for 3 N-glycans, 2 for 1 O-glycan) profile. Panel (e) shows relative abundance of predicted N-glycans and the relative similarity between EPO 113 vs EPO 112.a and EPO 112.b. Panel (f) shows predicted N-glycans (filled circles) of EPO-112a, EPO-112b and EPO-113 in the biosynthesis pathway. Precursor routes (dotted lines) to the predicted N-glycan biosynthesis shows different topological roots for EPO-112a & 113 vs 112b. * and ** nodes indicate the earliest divergence point for N-glycan biosynthesis that involves non-bisection or bisection, respectively.

FIG. 9: Panel (a) shows pre- and post-septic shock mass spectra of desialylated AACT overlayed shows an increase in overall average mass as well extent of glycan biosynthesis. Up to 9 additional LacNAcation events and 10 fucosylation events in total can occur. Panel (b) shows comparative desialylated AACT gp networks, with septic shock (blue) distributions showing a bimodal shift towards continued branching/elongation versus fucosylation. Panel (c) shows distributions of LacNAcation/branching events for different extents of fucosylation. Panel (d) shows N-glycan structures correlated to proposed pathways pre- and post-septic shock. Panel (e) shows glycan biosynthesis pathways for probable N-glycans shown in (d), with septic shock N-glycans diverging at the same topological root. Panel (f) shows, for all 10 patients, in silico prediction of glycans from gp networks show significant changes to specific predicted glycan features between the pre-sepsis (timepoint 1, tp1), onset-sepsis (ICU admittance) (timepoint 2, tp2), and recovery biospecimens (ICU discharge) (timepoint 3, tp3). Panel (g) shows Z-score analysis compares network correlations between pre-sepsis to the average across all pre-sepsis samples. ** The “pre-sepsis” samples for patients 4 and 6 were reported to be collected ˜3 weeks prior to onset of sepsis, compared to ˜2-5 days for others suggesting that signs of infection were already observable in patient samples several days prior to diagnosis of sepsis shock and admittance into the ICU.* Network data for Patient 2 indicated the onset of septic shock already occurred at the time of the “pre-sepsis” sample collection. Panel (h) shows assessment of ratios of the PNA predicted glycan #5 at tp1 to glycan 4 at tp2 correlates with expected number of days needed to recover and be discharged from the ICU (i.e., prediction of tp3).

FIG. 10: The comprehensive N-glycan bioprocessing pathways in humans with consideration for specific structural characteristics that dictate N-glycan growth. Panel (a) shows the pathway is distinguished based on the extent of antennary growth and bisection of the central mannose by GlcNAc addition. Panel (b) shows the pathway is distinguished by extent of fucosylation at either the core or periphery or both as well as the total number of sialic acids. Panel (c) shows the commonly observed “standard” N-glycan structures that terminate the N-glycan biosynthesis pathway. Non-standard N-glycans (not shown) would also incorporate peripheral fucosylation in bi-, tri- and tetra-antennary species.

FIG. 11: Glycoproteoform Centrality Score (GCS) optimization separates highly connected gps from unlikely counterparts. Filtering criterion is dependent on the highest differential in closeness centrality (inset plot), resulting in the removal of gprᵢ that do not contribute to the network fidelity.

FIG. 12: Predicted Pathway Optimization (PPO) for L-PGDS. Panel (a) shows a histogram of the calculated scores for all possible permutations (648,000 total) of all 15 predicted L-PGDS N-glycans from the intact data (axes in logarithmic scale). The optimal assignment of 15 N-glycans within the N-glycan pathway map was determined based on maximizing connectivity between all points in the map. *′**′*** Portions of non-optimal network paths of the same 15 N-glycans, where connectivity progressively increases with respect to score. Panel (b) shows network of the N-glycan database that highlights the N-glycans (nodes) that are associated with the optimized pathway (red) as well as all other N-glycans (green) that have a matching composition (to predicted L-PGDS N-glycans) but were not determined to be part of the optimal pathway (see *′**′*** (a)). For L-PGDS, Inventors calculated p<0.000016 for the most probable pathway (a near identical match to the pathway/structures observed in bottom-up studies (see FIG. 6d).

FIG. 13: Panel (a) shows the method for predicting the number of N-glycosylation sites from top-down glycan compositions. Panel (b) shows intensity-scaled bubble plot of matches for intact L-PGDS glycoprotein data. Panel (c) shows Pie charts show total intensity of matched gp data where each ring represents a different number of N-glycosylation sites (for L-PGDS, EPO and NIST mAb samples). Some EPO variants contain multiple LacNAc residues, which were collapsed down to the non-LacNAc variety prior to calculation.

FIG. 14: F₀₋₄∈LPGDS network for all gps observed for CSF L-PGDS.

FIG. 15: Panel (a) shows correlation comparison of MS data between different EPO variants and WT EPO. Panel (b) shows correlation comparison of predicted N-glycans and relative intensities between different EPO variants and WT EPO. In both, size and hue are proportional to degree of similarity between two datasets.

FIG. 16: Individual glycoproteform networks for AACT septic shock patient data from 4 different time points: t1—pre-septic shock, t2—onset of septic shock, t3—t2+1 day, t4—post surgical intervention. For each network, Δ[H+GN] additions are resolved vertically from bottom to top and fucosylation increases from left to right.

FIG. 17: Panel (a) shows predicted N-glycan distribution of healthy AACT (t1) and corresponding relative up or down-regulated N-glycans with respect to disease onset and progression (t2-t4). Panel (b) shows a volcano plot of gp show fold change against p-values, highlighting down regulated (right) and up regulated (left) gp with respect to healthy timepoint (t1) versus disease progression (t4). (Bottom) shows a bimodal transition for weighted-average glycan composition from healthy (purple) to disease (green and blue). Compositions in green represent F₀ and F₁ compositions (first septic shock pathway) while blue represents compositions with F₂₊ (second septic shock pathway).

FIG. 18: (Left) Calculation of glycoproteoform assignment based on mass accuracy and (Right) bootstrap aggregated ensemble modeling of the isoelectric point separation using a series of cost-weighted decision trees. Both methods provide independent likelihood values ranging from 0-1, with 1 being the maximum probability.

FIG. 19: Example of simulated annealing on glycoproteoform networks. Early iterations pick assignments at random, typically resulting in poorly connected network (black nodes). Gradual optimization via random alterations slowly improves the network connectivity (blue nodes) based on specific glycan mass shifts (F, H, GN, S), which is then optimized as T decreases.

FIG. 20: Panel (A) shows SDS-PAGE gel of Offgel IEF fractions collected on RNaseB. (B) LC-MS analysis helps show that pI separations help differentiate deamidated forms of 5 glycoproteoforms of RNAseB. Off-shoot subnetworks may be assigned on the pI axis with either 1 or 2 deamidation events. +1 Am or +2 deamidation events may be associated with a 1 Da and 2 Da increase in mass relative to the base forms (respectively).

### DETAILED DESCRIPTION OF THE INVENTION

Disclosed herein is proteoform network analysis (PNA). PNA unravels glycoprotein microheterogeneity by organizing glycoproteoforms into networks which contextualize the glycosylation machinery targeting the protein. Direct MS analysis of intact glycoproteins, such as by native and top-down mass spectrometry, produces highly complex yet information rich datasets. However, the non-templated biosynthesis of glycosylation necessitates interpretation strategies that currently are lacking. To overcome this bottleneck, PNA unravels glycoprotein microheterogeneity by automating gp network organization and subsequent quantification of glycosylation dynamics, e.g., in inflammatory responses or other changes in disease states. PNA provides robust predictive methods for site-independent characterization of glycosylation enzymology and putative glycan structural features. Hence, the disclosed technology allows for confident and rapid characterization of glycoproteins from healthy and disease-related biospecimens at the broader compositional and specific enzymological levels.

PNA is designed to analyze complex glycoprotein heterogeneity. The use of machine learning, network analysis, and graph theory on multiplexed gps obtained from intact protein MS screens from as little as 10-100 μL of biospecimen sample provides a rigorous data stream that will overcome many challenges associated with pre-analytical sample loss caused by conventional digestion of glycoproteins or release of their glycans. Network quantitation can capture the flux (intensity) of 100-1000s of gps thereby amplifying redundant monosaccharide-related alterations that are difficult to detect by analysis of individual glycopeptides or glycans. This approach is highly sensitive to subtle changes in protein glycan composition that will enable prospective predictions of etiology such as molecular events that drive glycosyltransferase or downstream consequences of glycosylation alterations on acute phase proteins (APP) function. This technology approach uses a robust glycan prediction method that mines the network data for plausible glycans present on the protein, resulting in a confident list of glycan targets that may be validated.

Through effective management of data dimensionality, glycoproteoform heterogeneity is efficiently probed for etiological insights related to glycan biosynthesis and (dis)similarity across samples. The Examples demonstrate the utility of the disclosed technology to probe insights in a biofluid (e.g., di-N-glycosylated, lipocalin-prostglandin d-synthase from cerebrospinal fluid), cell (e.g., tri-N-glycosylated, human recombinant erythropoietin variants), or disease-specific manner (e.g., penta-N-glycosylated α-1-antichymotrypsin from sepsis patients). The disclosed technology allows for identification of glycoproteoforms as diagnostic or prognostic biomarkers in clinical settings or targets for pharmacokinetic-pharmacodynamic modeling in protein-based drug development. Moreover, identification of biomarkers allow for the treatment determinations to be made for the subject.

Glycosylation occurs on approximately half of all proteins, serving to modulate protein interactions, drug (biotherapeutics or biosimilars) activity and stability, or serve as biomarkers for diseases such as cancer and neurodegenerative diseases. N-glycosylation is one of two abundant forms of glycosylation N-glycans are attached to the side chain of an asparagine present in a consensus sequence (Asn-X-Ser/Thr, where X≠Pro) and derived from biosynthesis reactions where mannosidases (Man), and N-acetylglucosaminyl- (GnT), fucosyl- (FucT), galactosyl-(GalT), and sialyl- (SiaT) transferases sequentially add (or remove) monosaccharides from specific glycosidic bonds.

The order and concentration of enzymes, substrates, and reaction rate k_{cat}/Kₘ create a large repertoire of glycans (e.g., high mannose, hybrid, or complex (bi-, tri-, tetra antennary)) that gives rise to a complex set of possible glycoproteoforms (gps) for a given glycoprotein (G) because microheterogeneity scales exponentially with the number of glycosylation sites and is further exacerbated by N-glycan branch elaboration such as fucosylation, phosphorylation, sulphation, or poly-LacNAc (Galβ1-4GlcNAc) extensions.

Characterization of N-glycan composition, structure, or position within a target protein, or across a proteome, may be accomplished by various glycoproteomics techniques. Commonly, the workflows either release glycans by PNGase F or digest the protein into glycopeptides, followed by chromatographic separations, accurate mass determination, and sequential fragmentation of the peptide or glycan backbones by tandem mass spectrometry (MS/MS). On the other hand, intact protein MS, such as with top-down MS (TDMS) or native MS (nMS), is advantageous because the analysis is on gps directly, providing a direct measurement of their relative ratios by avoiding the tedious peptide digestion or glycan removal steps. However, direct gp analysis is less favored since the determination of the number, position, composition, and anomeric structures of glycans is often not readily possible from intact MS data alone. Plus, data mining is challenged by the potentially hundreds of co-occurring spectral gps whose hierarchy are often not contextualized relative the underlying glycosylation machinery.

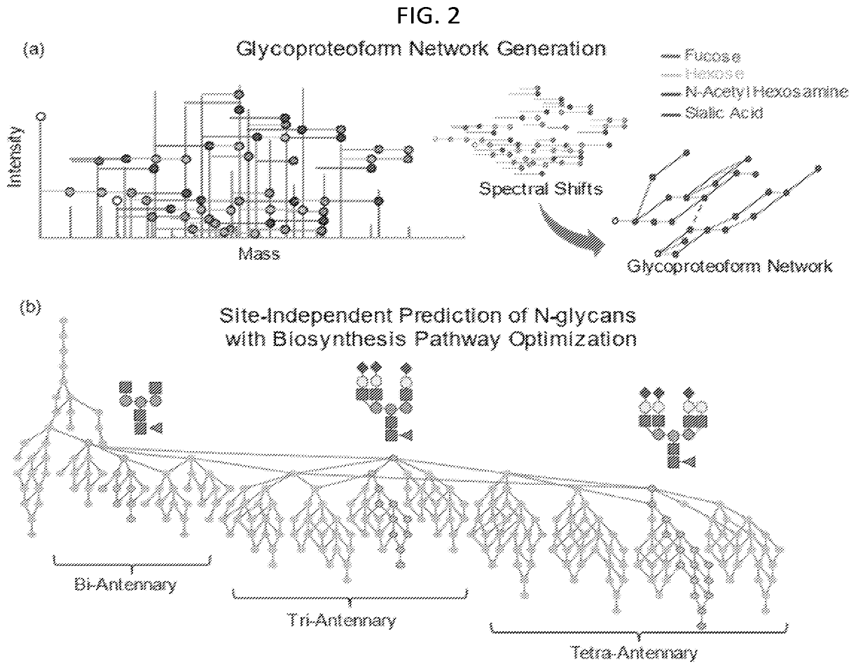

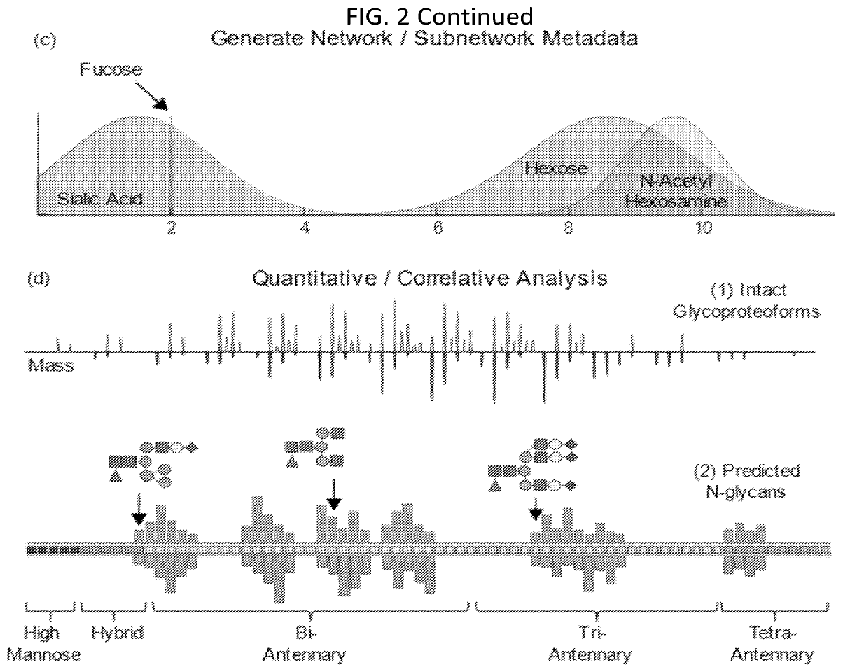

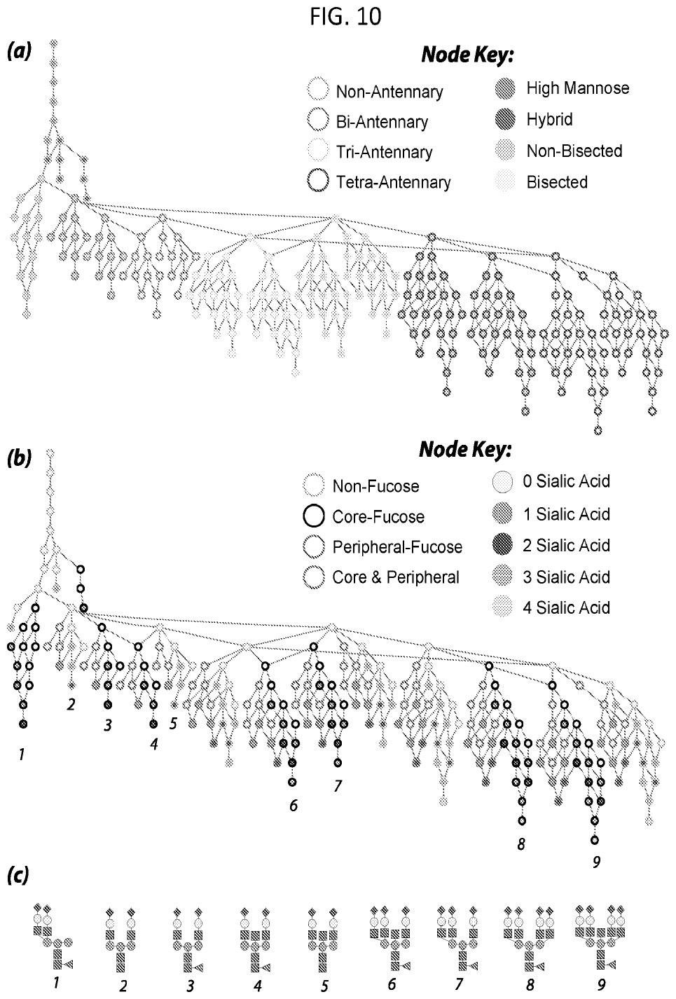

Proteoform network analysis (PNA) overcomes the limitations of the art and allows for the multiplexed visualization and characterization of complex gp relationships (FIG. 2). Glycoproteoform networks may be constructed based on separation of mono- or poly-saccharide features (FIG. 2a) followed by a site-independent prediction of probable N-glycans mapped to biosynthesis pathways that permit inference of topology features (e.g., high-mannose; bi-, tri-, tetra-antenna; GlcNAc bisection) (FIG. 2b and FIG. 10). Various metrics can be generated to help quantify network or subnetwork attributes and discern atypical glycan-level features not readily ascertained from any single gp (e.g. branch elongation or O-glycosylation) (FIG. 2c-2d). The workflow's robustness is demonstrated via re-analysis of previously collected TDMS and nMS gp data (Table 2), including di-N-glycosylated L-prostaglandin D-synthase (L-PGDS), human recombinant tri-N-glycosylated erythropoietin (rhEPO) variants, and penta-N-glycosylated α-1-antichymotrypsin (AACT)), and were validated against glycan data obtained from conventional bottom-up or middle-down glycoproteomics.

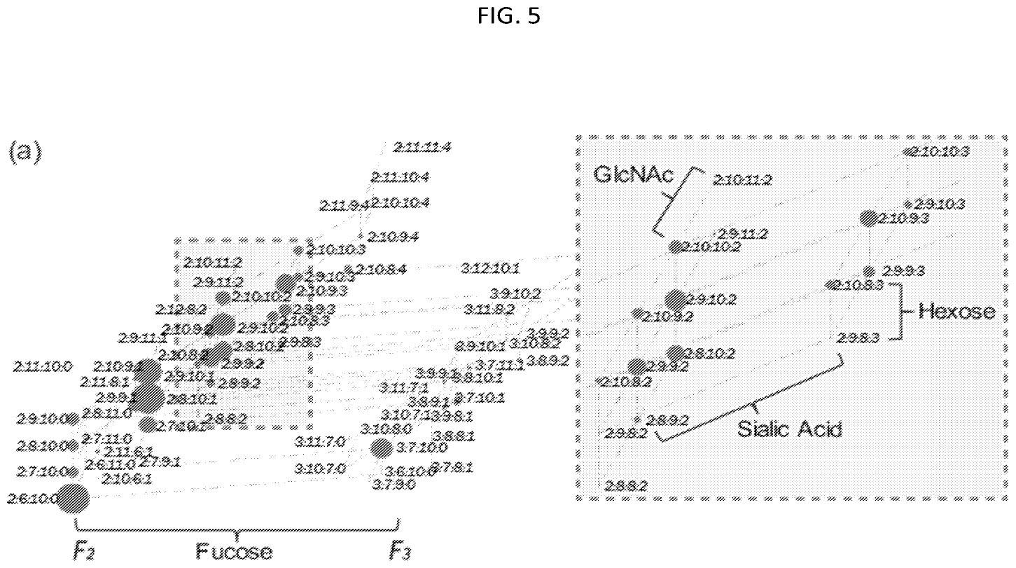

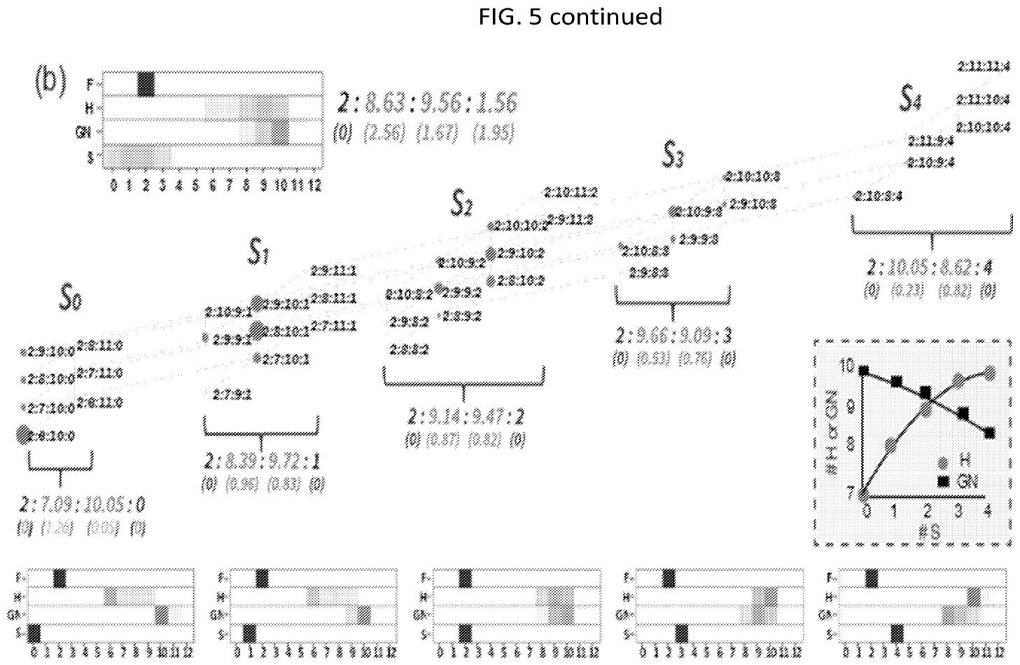

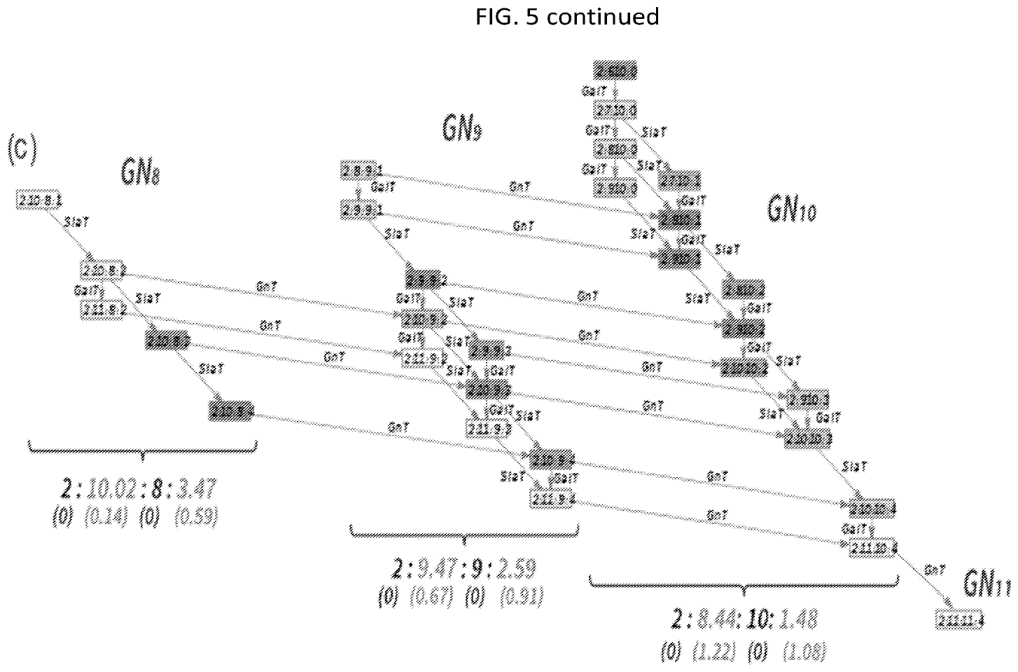

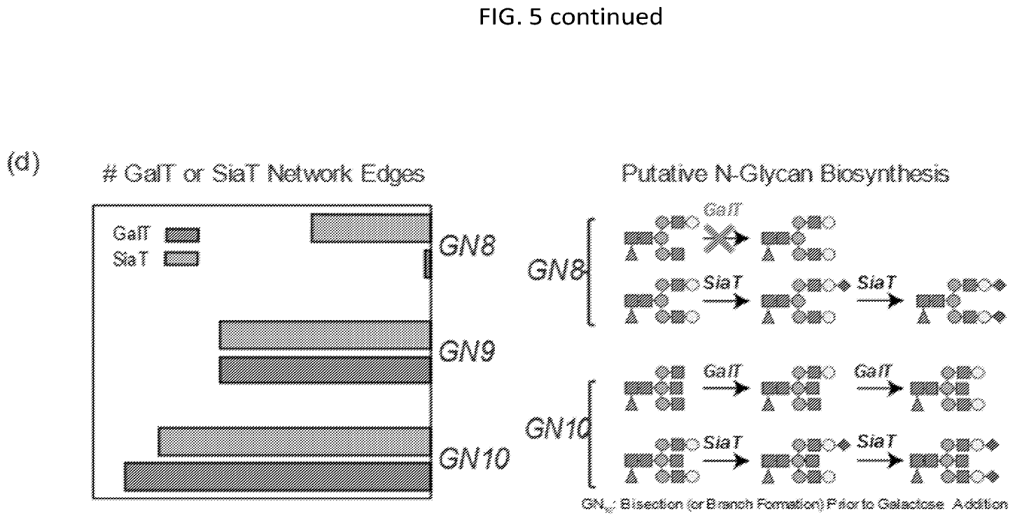

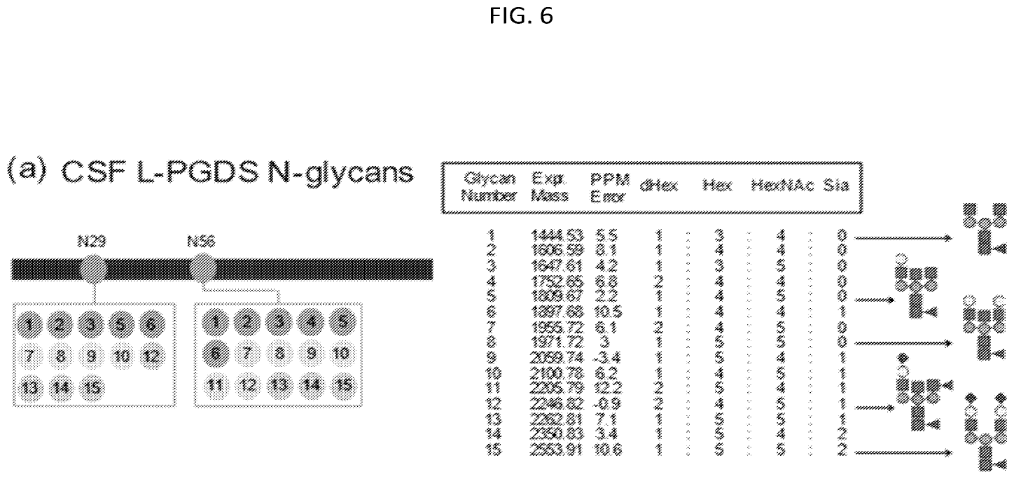

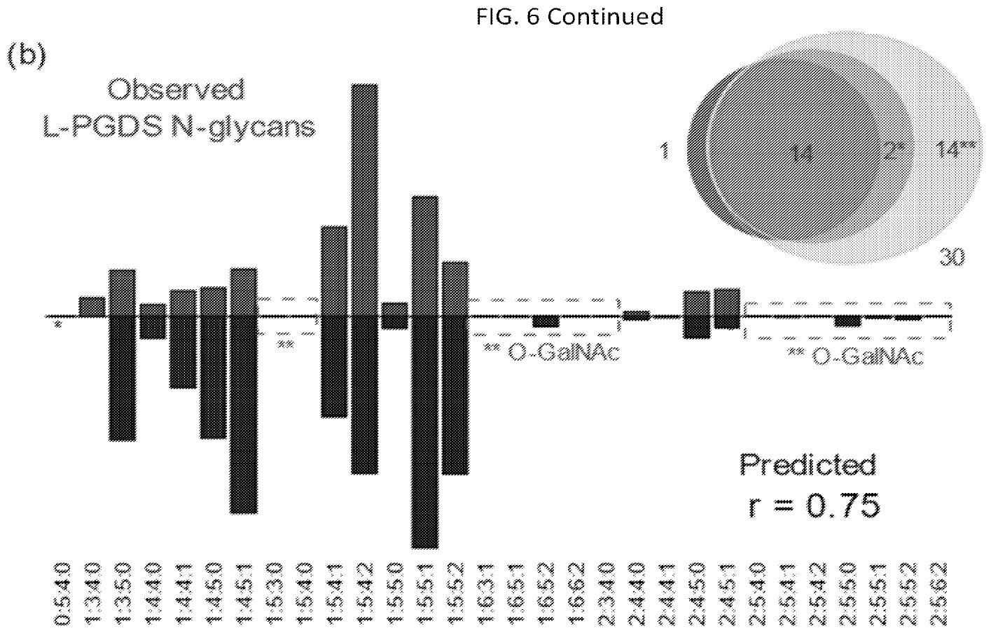

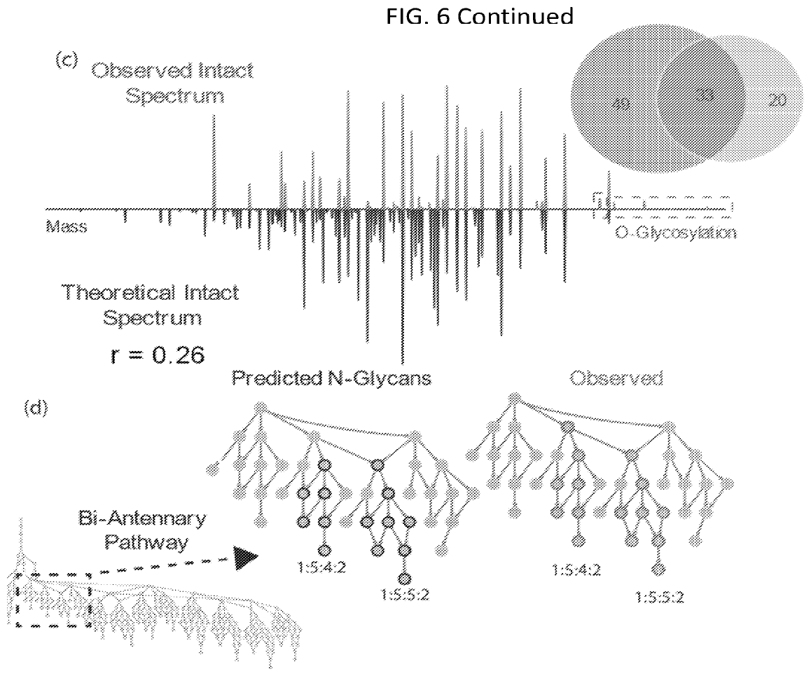

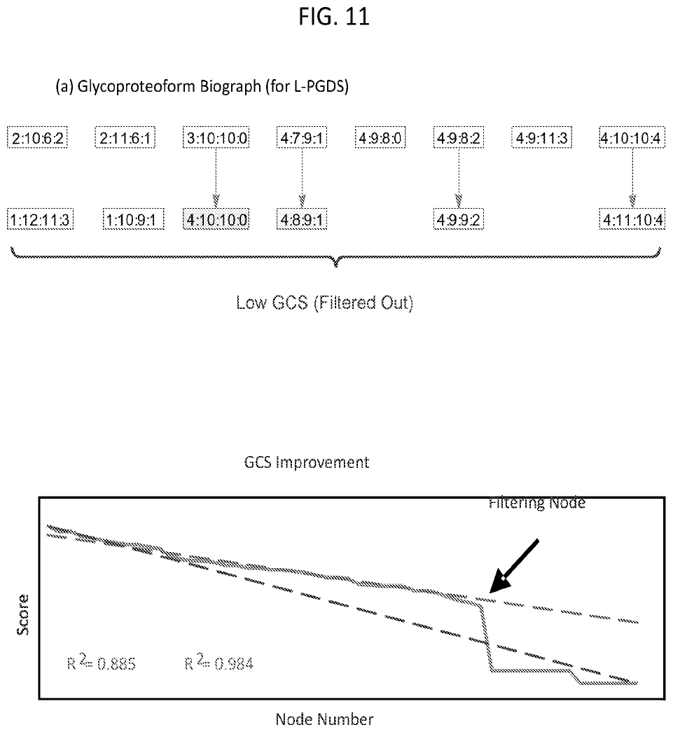

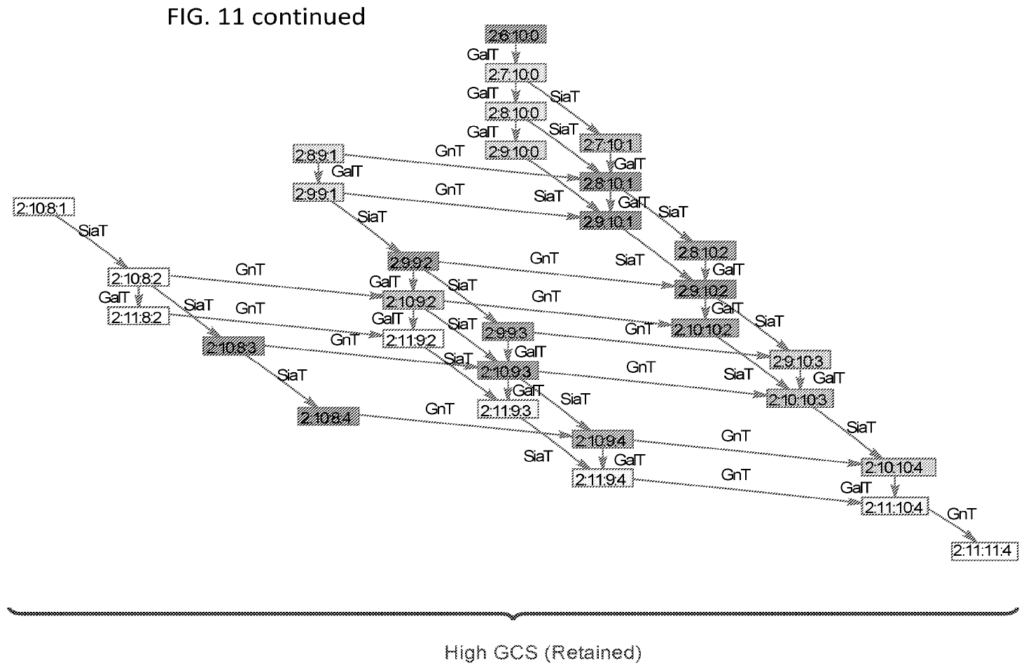

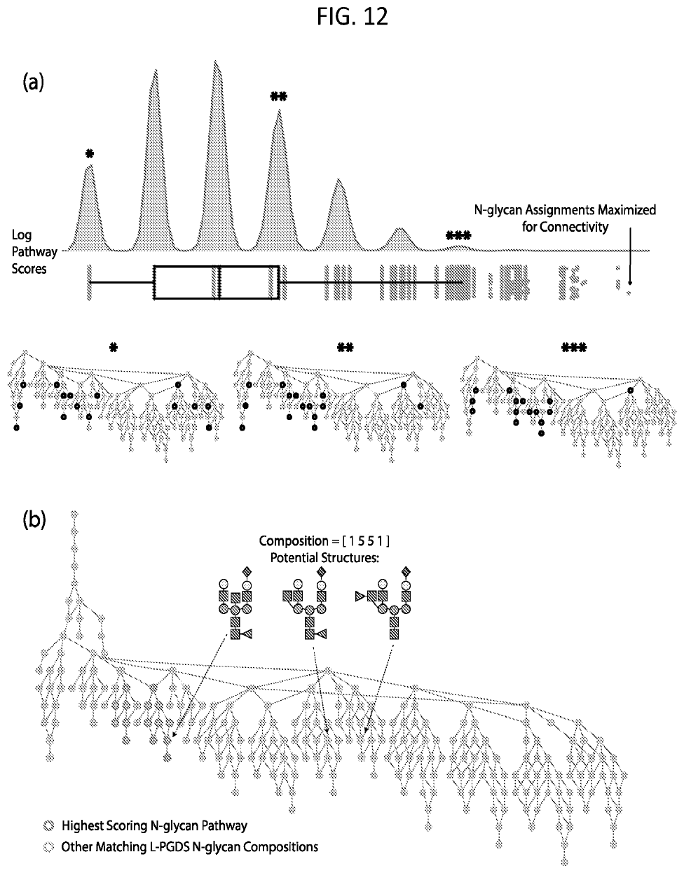

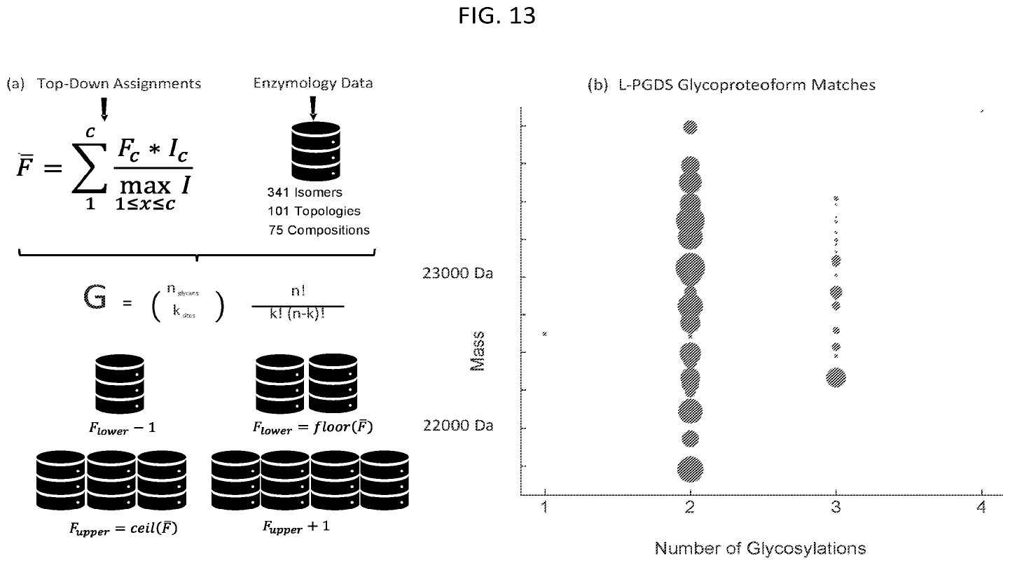

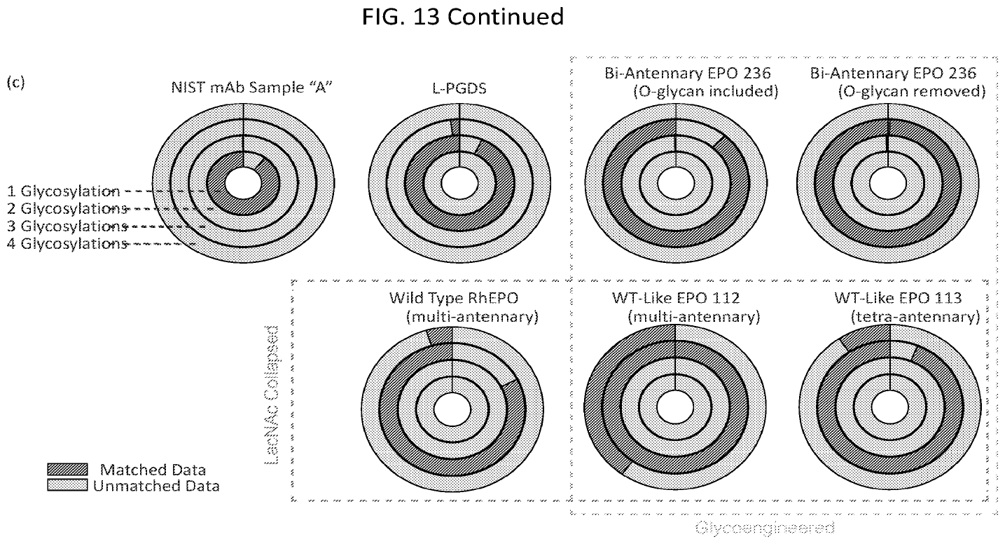

The technology may be better understood with the following nomenclature. A set of gps for a glycoprotein (gps∈G) are described by their intensity weighted fucose (F), hexose (H), N-acetyl hexosamine (GN) and sialic acid (S) content (denoted in a 4-sequence number system -F:H:GN:S). The directional or biographical networks generated consisted of nodes (i.e. gps) and edges which link spectral gps to others of the same G based upon stepwise addition of common glycan moieties (e.g. F, H, GN, S, LacNAc, etc.) (e.g., FIG. 5-6). PNA can automatically optimize network topologies to maximize differentiation of gp heterogeneity and eliminate potentially erroneous compositional assignments, e.g., via centrality-discriminant scoring method (Glycoproteoform Connectivity Score, GCS) (FIG. 11). PNA also estimates the number of glycosylation sites (NSG) (FIG. 13) and makes site-independent predictions of the abundance of plausible N-glycan compositions based on a novel probable pathway optimization (PPO) step (FIG. 12).

Glycoprotein (G) means a translated gene product subjected to one or more 1 glycosylation events at one or more 1 amino acid residue(s).

Glycoproteoform (gpᵢ) means a specific form of G with unique amino acid (aa) sequence, total F:H:GN:S composition, or other post-translational modifications (PTMs) that lead to a distinct intact mass when measured by mass spectrometry. (gp_{i mass}=aaₘₐₛₛ+F:H:GN:Sₘₐₛₛ+PTMₘₐₛₛ)

(gp∈G) means a set or subset of glycoproteoforms for glycoprotein G.

F:H:GN:S or F:H:GN:S * means the compositional polysaccharide isobar composed of differing number of fucose (F), hexose (H), n-acetyl hexosamine (GN) and sialic acid (S) associated with either individual glycans (e.g., N-glycans) or the sum of the core compositional components of all glycans present on the Gp backbone. * F:H:GN:S is italicized when referencing a gp. F:H:GN:S is not italicized when referencing a glycan.

N-glycan means an Asn-linked (N-linked) oligosaccharide of a high mannose, hybrid, or complex type.

Gp_{N-MAX} means the maximum composition in a network/subnetwork that derive from contributions from only standard N-glycans including high-mannose, hybrid, or bi-, tri-, or tetra-antenna structures that may or may not be sialylated or contain core-fucose.

N-Glycan_{Max} means the most processed N-glycan that exists on the target glycoprotein after PPO. This value is adjustable depending on prior knowledge of glycoprotein source, species or expected N-glycosylation termination point.

Poly LacNAc means a repeating N-acetyllactosamine unit consisting of a Galβ1-4GlcNAc that occurs on N-glycan galactose termini.

O-glycan means a Ser- or Thr-linked (O-linked) oligosaccharide.

Core 1 O-glycan means the addition of Galβ1-3GalNAc to a serine or threonine residue that can have additional transferase processing or elongation followed by termination via sialic acid addition.

Glycoproteoform or glycan composition means the raw number of 4 specific sugar residue types, e.g., fucose, hexose, N-acetyl hexosamine, and N-acetylneuraminic acid, that make up a glycoproteoform or glycan.

Glycan topology means the ordered arrangement of specific glycan residues which indicate a covalent bond exists between two sugar residues but with no specificity with regards to bond position or orientation.

Glycan structure means the specific arrangement of glycans bonded to one another including specific isomeric and anomeric forms.

Here, a glycoprotein (G) is a gene-level distinction of a familywise reference for a set of closely related translated product(s) of a single gene exclusive of descriptors of sequence-level changes (e.g., polymorphisms) and modification-level microheterogeneity (e.g., type, number, or position of PTMs). Aglycan (N- or O-) is a polysaccharide-level distinction that references a sugar-composition or topology that does not differentiate branching, anomeric features, or correlate to a specific G. For example, N-glycan compositions within the biosynthesis pathway map (FIG. 2b and FIG. 10) are denoted by (non-italicized) F:H:GN:S; where F, H, GN, and S=the number of fucose, hexose (mannose or galactose), N-acetyl glucosamine, and sialic acid, respectively. Finally, a glycoproteoform (gpᵢ) refers to a specific (i-th) form of G identified from experimental mass spectrometry datasets. Each gp, will exist with either unique amino acid sequence (aa), total glycan-composition (denoted at the gp-level by an italicized F:H:GN:S), or PTM features that lead to a measurable intact mass (gpᵢ₋ₘₐₛₛ=aaₘₐₛₛ+F:H:GN:Sₘₐₛₛ+PTMₘₐₛₛ). For proteoforms harboring multiple glycans at different sites, the sugar-composition of gpᵢ is contextualized after the sum of the core sugar components of all glycans regardless of position into a single generic F:H:GN:S composition. For example, for a hypothetical di-glycosylated protein harboring two 1:5:5:2 N-glycans, gpᵢ=2:10:10:4.

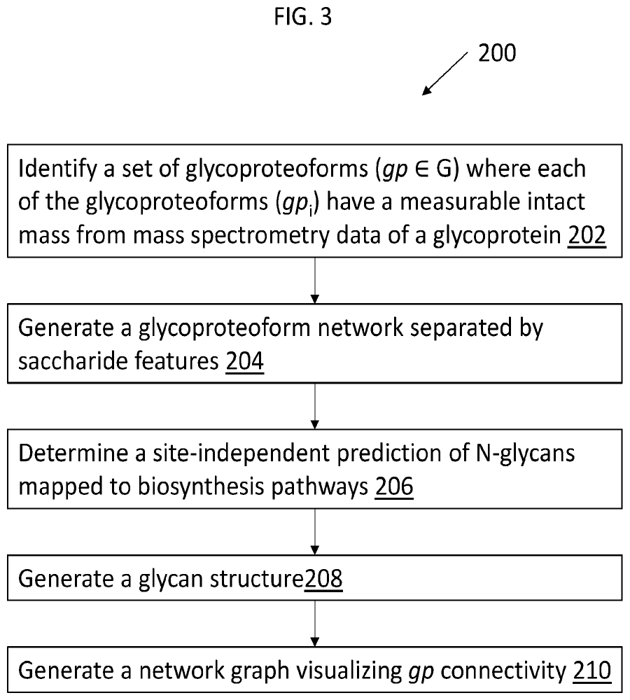

Referring to FIG. 3, a flowchart is provided as setting forth the steps of an example method 200 for determining a glycan structure of a glycoprotein in accordance with the present disclosure. The method 200 includes identifying a set of glycoproteoforms (gp∈G) where each of the glycoproteoforms (gpᵢ) have a measurable intact mass from mass spectrometry data of a glycoprotein, as indicated at step 202. The mass spectrometry data may have been previously acquired and provided to a computer system from a memory or other data storage device, or may including acquiring a mass spectrum using a mass spectrometry unit and communicating the acquired data to a computer system, which may form a part of the mass spectrometry unit.

The method 200 includes generating a glyoproteoform network separated by saccharide features, as indicated in step 204. The networks connections are derived from gp assignments (F:H:GN:S). Each gpᵢ is iteratively given the possibility to connect to an adjacent node based on mass shifts of F, H, GN or S and will form an edge in the network only if the corresponding gpᵢ+F (or H, GN, S) also exists in the mass spectrum data. Each gpᵢ carries out the same iterative operation until all nodes and corresponding edges are determined. The formation of a biograph requires the creation of a sparse pairwise matrix where M is an N-by-N matrix where N is the total number of unique compositional gp in the mass spectra. M is a zeros matrix with logical values of 1 when two gp at their respective index, Mᵢⱼ have a connecting node on the network.

The method 200 includes determining a site-independent prediction of N-glycans mapped to biosynthesis pathways, as indicated in step 206. F:H:GN:S compositions for each gpᵢ∈G network were used for site-independent predictions of probable standard N-glycan F:H:G:S compositions and intensity.

The method 200 includes generating a glycan structure, as indicated in step 208. Intact gp data is used to predict a site-independent N-glycan compositional distribution based on all gp observed in the network.

The method 200 may optionally include generating a network graph visualizing gp connectivity, as indicated in step 210. Network graphs provide concise visualization of topological order or connectivity of complex data and facilitate determination of properties that describe or quantify the network, sub-structures, or individual nodes/edges.

In some aspects, the method 200 includes preprocessing the mass spectrum of the glycoprotein. Preprocessing the mass spectrum may include, but is not limited to protonating all the peaks in the spectrum, performing a baseline correction, spectral alignment of profiles, normalization, peak preserving noise reduction, peak finding with wavelet denoising, binning through peak coalescing and combinations thereof. In some aspects, the method 200 includes preprocessing the mass spectrum to identify and add in computed complementary peaks missing from the mass spectrum. In some aspects, the method 200 further includes matching mass spectrum peaks in the mass spectrum with theoretical mass spectrum peaks of a theoretical spectrum of the molecule. The method 200 may further includes producing a filtered mass spectrum of the glycoprotein by removing unmatched mass spectrum peaks from the mass spectrum.

The methodology for performing method 200 are further described below.

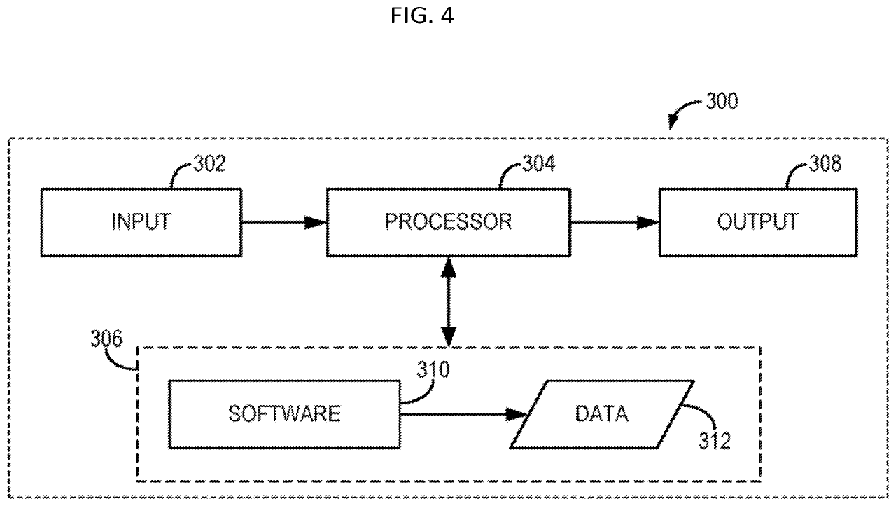

Referring now to FIG. 4, a block diagram of an example of a computer system 300 that can be used to implement the methods described herein and, specifically, determine a topology or molecular formula for a molecule using mass spectrometry data. The computer system 300 generally includes an input 302, at least one hardware processor 304, a memory 306, and an output 308. Thus, the computer system 300 is generally implemented with a hardware processor 304 and a memory. In some embodiments, the computer system 300 can be implemented, in some examples, by a workstation, a notebook computer, a tablet device, a mobile device, a multimedia device, a network server, a mainframe, one or more controllers, one or more microcontrollers, or any other general-purpose or application-specific computing device.

The computer system 300 may operate autonomously or semi-autonomously, or may read executable software instructions from the memory 306 or a computer-readable medium (e.g., a hard drive, a CD-ROM, flash memory), or may receive instructions via the input 302 from a user, or any another source logically connected to a computer or device, such as another networked computer, server. The input 302 may take any shape or form, as desired, for operation of the computer system 300, including the ability for selecting, entering, or otherwise specifying parameters consistent with operating the computer system 300.

In general, the computer system 300 is programmed or otherwise configured to implement the methods and algorithms in the present disclosure, such as those described with reference to FIG. 3. For instance, the computer system 300 can be programmed to generate a glycan structure for a glycoprotein based on experimental mass spectrometry data. In some aspects, the computer system 300 may be programmed to access acquired data from a mass spectrometry unit, such as mass spectroscopy data that includes mass spectrum peaks corresponding to a precursor ion and fragment ions. Alternatively, the mass spectrum may be provided to the computer system 300 by acquiring the data using a mass spectrometry unit and communicating the acquired data to the computer system 300, which may be part of the mass spectrometry unit.

The computer system 300 may be further programmed to process the mass spectrum to generate a glycan structure for the glycoprotein. The computer system 300 may identify a set of glycoproteoforms (gp∈G) where each of the glycoproteoforms (gpᵢ) have a measurable intact mass, generate a glycoproteoform network separated by saccharide features, determine a site-independent prediction of N-glycans mapped to biosynthesis pathways, generate the glycan structure, or any combination thereof from mass spectrometry data of an glycoprotein or, more particularly, an intact glycoprotein.

The input 302 may take any suitable shape or form, as desired, for operation of the computer system 300, including the ability for selecting, entering, or otherwise specifying parameters consistent with performing tasks, processing data, or operating the computer system 300. In some aspects, the input 302 may be configured to receive data, such as data acquired with a mass spectrometry unit, a database, or a combination thereof. Such data may be processed as described above to generate a topology for the molecule of interest. In addition, the input 302 may also be configured to receive any other data or information considered useful for determining the topology of the molecule using the methods described above.

Among the processing tasks for operating the computer system 300, the one or more hardware processors 304 may also be configured to carry out a number of post-processing steps on data received by way of the input 302. For example, the processor 304 may be configured to generate a network graph visualizing gp connectivity for the glycoprotein, disease onset in a subject, or disease recovery in a subject using experimental mass spectrometry data. The processor 304 may be configured to implement the same or similar method tasks as described in FIG. 3.

The memory 306 may contain software 310 and data 312, such as data acquired with a mass spectrometry unit, and may be configured for storage and retrieval of processed information, instructions, and data to be processed by the one or more hardware processors 304. In some aspects, the software may contain instructions directed to processing the input mass spectrum or mass spectroscopy data to be processed by the one or more hardware processors 304. In some aspects, the software 310 may contain instructions directed to processing the mass spectroscopy data or mass spectrum in order to generate a glycan structure of the glycoprotein, as described in FIG. 3. The software may also contain instructions directed to generating generate a network graph visualizing gp connectivity for the glycoprotein. In some aspects, the software may also contain instructions directed to identify disease onset in a subject or disease recovery in a subject using experimental mass spectrometry data.

It is to be appreciated that alternative mass spectrometry units may be used in accordance with the present disclosure. In general, any mass spectrometry unit capable of ionizing chemical species and separating them based on their mass-to-charge ratio may be used in accordance with the present disclosure. Suitable examples may include FTMS, MD MS, EMR MS, TDMS, AMS, GC-MS, LC-MS, ICP-MS, IRMS, MALDI-TOF, SELDI-TOF, Tandem MS, TIMS, SSMS, and similar mass spectrometry instruments

To solve the challenges facing TD analysis on glycoproteins, PNA facilitates the assignment of glycoproteoforms when the parent glycoprotein harbors one or more than one N-glycosylation site. PNA is a probability-driven assignment of glycan features (F:H:GN:S composition) on a protein (glycoproteoforms) from accurate mass matching of theoretical mass values derived from a glycan database against mass spectral data. Generative models to assess assignment likelihoods are established through (1) singular or multidimensional separation targeting specific glycan features; (2) machine learning of separation techniques to improve predictive outcomes and/or (3) simulated annealing to optimize computationally bottlenecked glycoprotein data.

Chromatographic separations may help resolve glycoproteoforms. This may facilitate the glycoprotein analysis. Glycoproteoforms can be separated using various chromatographic techniques which leads to even more complicated set of data. IEF-LC-MS permits sensitive, high dynamic range, and reproducible measurements on intact proteins (>200 kDa) from cell lysates, biofluids, and tissues. IEF fractions are collected at intervals across an immobilized pH gradient (IPG) (e.g., 24 fractions from pI 3-10) prior to LCMS. IEF-SPLC-MS results are conceptually similar to 2D SDS-PAGE (2DGE) in that proteins are subjected to isoelectric point (pI) separation prior to mass analysis. However, FTMS provides exceedingly high mass resolving power that differentiates closely related proteoforms. For example, CSF analysis led to the discovery >200 lipocalin-type prostaglandin d-synthase (L-PGDS) glycoproteoforms (FIG. 3), an ˜36× improved detection efficiency compared to 2DGE.

Separation targeting specific glycan features is a method that uses accurate mass alone or accurate mass in combination with some chromatography separation characteristic to arrive at the F:H:GN:S composition.

Machine learning is used to create a predictive model of expected experimental pI based on all other experimental features (mass, glycan composition, intensity, etc.).

Simulated annealing selects the best glycoproteforms from a large volume of possible glycoproteoform assignments based on established glycan patterns that are in the mass spectrum.

An aspect of this technology is to provide an identity (descriptor) to each glycoproteoform. This includes assigning the appropriate number of specific sugar residues to each peak in the mass spectrum. However, as noted in the definition of a glycoproteoform the descriptor could be expanded to include other information such as other PTMs or perhaps sequence related variants (e.g., mutations, SNPs, etc.). Alternatively, other features such as mass, chromatography characteristics, etc. may be used as well.

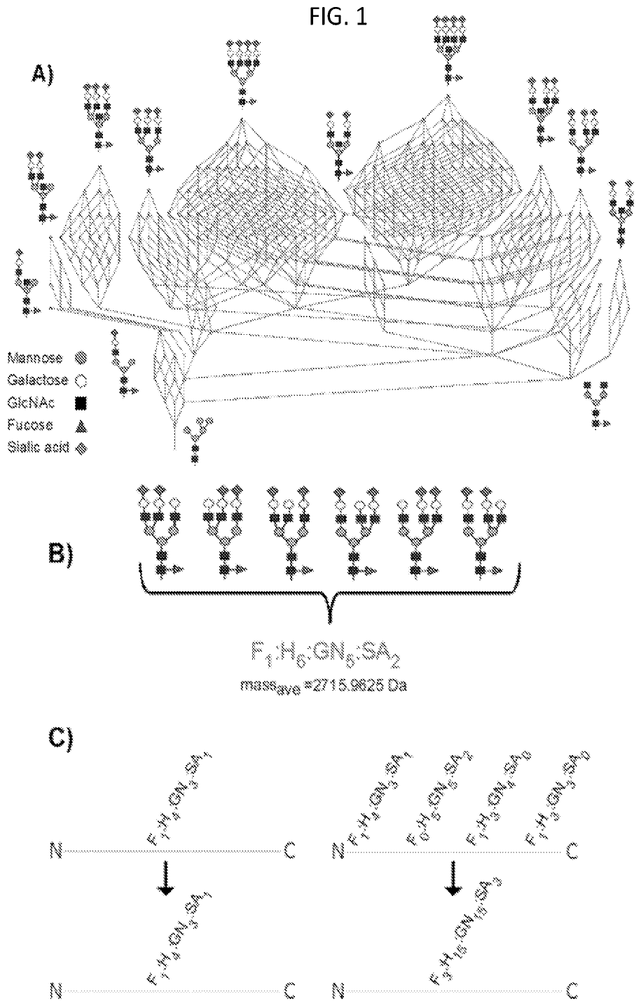

The sugar description starts with understanding that glycans exist in networks of sequential biosynthesis reactions (FIG. 1a). The most common glycans are formed by mannosidases (Man), and N-acetylglucosaminyl- (GnT), fucosyl- (FucT), galactosyl- (GalT), and sialyl- (SiaT) transferases. The flux of N-glycan intermediates within biosynthesis paths can be visualized through branched tree plots. While such plots highlight “glycan-level” microheterogeneity, for interpretation of mass spectrometry (e.g., MS1) datasets (such as for intact glycoproteins) one must consider the glycan's mass which is derived from the core sugar components: F:H:GN:S where F, H, GN, and S are the number of events of fucosyl (F, Δ146 Da), GlcNAc (GN, Δ203 Da), and sialic acid (SA, Δ291 Da), respectively. In some instances, n may be used to represent the number of events of fucosyl (F, Δ146 Da), GlcNAc (GN, Δ203 Da), and/or sialic acid (SA, Δ291 Da). Mannose and galactose are isobaric (i.e. same mass but different chirality) and are represented by hexose (H, Δ162 Da). Inventors note that consideration of only the mass of F:H:GN:S compositional isomers greatly simplifies microheterogeneity for MS1 level interpretation. For example, to generate databases from 335 common N-glycans (simple, hybrid, and complex up to tetra-antennary) only 70 F:H:GN:S compositional isomers have unique mass (FIG. 1b). For glycoproteins harboring multiple N-glycans, the descriptor model sums the core compositional components of all N-glycans into a single generic glycoproteoform (gₚ) with mass F:H:GN:S+p, where p=mass of the amino acid sequence (FIG. 1c). Data in Table 1 highlights some general database statistics for theoretical glycoproteoforms enumerated for a glycoproteins modified at 1-4 sites. Notably, this model predicts that mass spectrometry (e.g., MS1) level interpretation will be the same for all N-linked glycoproteins. One can use databases in different ways and their construction can vary based upon prior knowledge related to specific proteins, cells, tissues, organisms, etc.

TABLE 1
gp databases.

| # of | # of | Smallest | | Largest | |
|---|---|---|---|---|---|
| N- | unique | Glycoproteoform | | Glycoproteoform | |
| glycan | F:H:GN:SA | F:H:GN:SA | Massₐᵥₑ | F:H:GN:SA | Massₐᵥₑ |
| sites | compositions | compositions | (Da) | compositions | (Da) |
| 1 | 70 | 0:3:3:0 | 1095.99 | 1:1:7:4 | 3868.49 |
| 2 | 555 | 0:6:6:0 | 2191.98 | 2:14:14:8 | 7736.98 |
| 3 | 2152 | 0:9:9:0 | 3289.97 | 3:21:21:12 | 11605.47 |
| 4 | 5905 | 0:12:12:0 | 4383.96 | 4:28:28:16 | 15473.96 |

To successfully annotate glycoproteoforms from mass spectrometry data, one must consider the mass accuracy required to uniquely assign a glycoproteoform. For example, for the gp databases (Table 1), the mass differences between each proteoform reveals that except for mono-glycosylated proteins, a mass tolerance <1 Dalton (Da) is necessary to uniquely assign all gp (not shown). This may be analytically challenging in LCMS screens where large proteins are often observed without resolution of carbon-12 (¹²C) carbon-13 (¹³C) isotopes. Plus, annotation of glycoproteoforms is challenged by off-by-1 Da error shifts that could be due to both improper data deconvolution as well as glycoproteoform misassignment stemming from the small mass differences such as those associated with glycans harboring 2 fucose vs 1 sialic acid (Δm=1.02 Da).

To address this, databases may be stratified by Sn content of each gp. This rationale is that IEF or other separation techniques can separate glycoproteins into charged isomers that consist of glycoproteoforms with distinct SAₙ content at different pI. The SAₙ criteria of our databases showed that even for tetra-glycosylated proteins the mass tolerance to successfully annotate any species is >2.2 Da, which is readily achieved under low resolving power conditions on common mass spectrometers. Thus glycoproteins, harboring a multiplicity of N-glycan sites (e.g., 1, 2, 3, 4, or more than 4 N-glycan sites) and observed under both high resolving power (resolved carbon-12 (¹²C) carbon-13 (¹³C) isotopes) or low resolving power conditions, may be assigned by matching their mass and observed pI to those in theoretical gp databases if mass accuracy of <2 Da can be achieved.

In some embodiments, Fᵢ:Hⱼ:GNₖ:Sₗ may be assigned by accurate mass alone. In other embodiments, assignment of Fᵢ:Hⱼ:GNₖ:Siₗ by accurate mass in combination with chromatographic separations characteristics (such as IEF, HILIC, CE). For methods employing isoelectric focusing, one can predict theoretical gp pI values in an algorithmic manner that estimates a gp's pI from the pKa of the respective amino acids and the sugar components of the glycan or through machine learning approaches to provide likelihood of a gps' pI value versus its compositional assignment.

To capitalize on the information rich datasets generated by intact protein MS, PNA allows for the multiplexed visualization and characterization of complex gp relationships (FIG. 2). Glycoproteoform networks were first constructed based on separation of mono- or poly-saccharide features (FIG. 1a) followed by a site-independent prediction of probable N-glycans mapped to biosynthesis pathways that permit inference of topology features (e.g., high-mannose; bi-, tri-, tetra-antenna; GlcNAc bisection) (FIG. 1b). Various metrics may be generated to help quantify network or subnetwork attributes and discern atypical glycan-level features not readily ascertained from any single gp (e.g. branch elongation or O-glycosylation) (FIG. 1c-1d).

Datasets associated with individual IEF fractions may be binned using software to avoid duplicate reporting of redundant masses observed in adjacent IEF fractions by providing each unique mass value a summed total intensity and weighted pI value. The software may perform binning via a user defined tolerance such as time, pI units, intensity, and high and low resolution, respectively. For example, the user may define a binning criteria of ±3 min, ±3 pI units, >1,000 intensity, and 30 ppm and 2 Da for high and low resolution respectively. One can include an off by 1 Da test to help address common data processing error occurrences. Statistical approaches may also be employed to determine the likelihood that peaks should be binned.

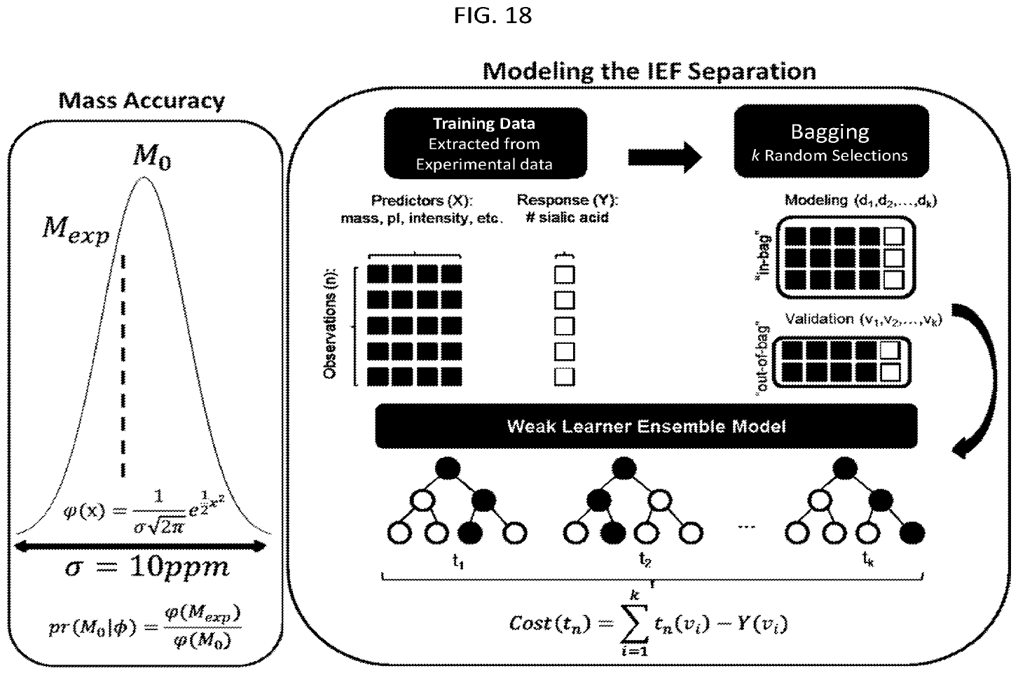

Off gel IEF is a robust method for separation of glycoproteins by their sialic acid content. However, run to run variability can occur, resulting in varied pI estimates for a given gp. To help overcome this source of error within the workflow, enabling accurate assignment of gp sugar compositions, a predictive model using the random-forest tree-bagger classification algorithm can me used. The model will be trained on high-intensity (e.g., >90% rel. ab.), high-mass accuracy (e.g., <1 ppm) gps in a given dataset that may have also been validated via bottom-up glycopeptide analysis. The training data consists of numerous experimentally observed factors (e.g., mass, fucose, hexose, n-acetyl hexosamine) which are ancillary factors in helping establish a framework for determining expected pI ranges for specific number of sialic acid residues. Multiple “weak learner” decision trees can be generated and then given an accuracy (i.e. “cost”) based on a randomized validation set. An “ensemble” model is generated from iteratively producing weak learners (˜300) and then uses cost-weighted ranking to produce the final model. Compared to other algorithmic models that have attempted to link pI and proteins, the ensemble learners offer the advantage of being highly specific and sensitive to the analytical platform performing the separation (FIG. 18).

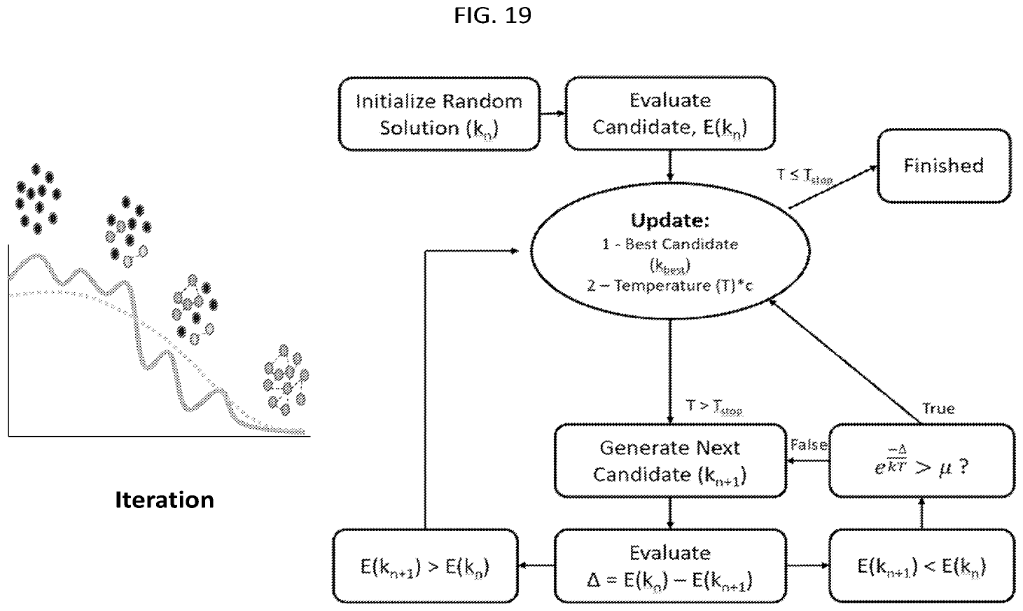

Large glycoproteins (>30 kDa) are often difficult to measure at high resolution (<5 ppm) in the Fourier transform setting of mass spectrometers. Typically, deconvolution of the data yields non-isotopic mass resolution and requires higher tolerance for compositional assignment. Single peaks are at risk of being assigned to multiple gps, and in highly heterogeneous scenarios, selecting the optimal set of gps (i.e. maximum connectivity) is impractical manually. Likewise, as mass tolerance or number of glycosylation sites increases, brute-forcing of all assignment combinations is also computationally unfeasible. Simulated annealing, a constraint-optimization method, addresses these challenges. Here, simulated annealing allows for rapid and automated discovery of the most interconnected gp assignments and is applicable at any resolution. Each mass value that contains multiple possible assignments is given one gp assignment at random and then scored based on the interconnectivity to the rest of the gps (also assigned at random). The process of random assignment and scoring continues, with the highest scoring set of gps being stored. A set temperature (T) will be used to gauge to degree of randomness introduced into the next iteration of assignments. As the number of iterations increased, T is gradually decreased due to an increased probability that a higher scoring gp set is correct (FIG. 19). Cooling T results in diminished changes of the gp assignment while offering small optimizations to the overall gp network. This method is designed to promote large changes in the gp assignment profile at first to find an initial starting point of highly interconnected species, and then gradually iterate on the best-scoring set (via cooling) to converge upon the most probable answer in a short time span.

Network graphs provide concise visualization of topological order or connectivity of complex data and facilitate determination of properties that describe or quantify the network, sub-structures, or individual nodes/edges. Here, networks and associated properties will be generated to assess transitive pathway(s) between the least to most enzymatically processed gps observed in MS data with different topological arrangements applied to resolve gp subsets with shared oligosaccharide features (e.g., F, H, GN, S, LacNAc, etc). With this approach glycosylation dynamics are expected to be captured in networks, allowing for detection of specific glycosyltransferase-dependent activity with respect to changes in disease state on a relative level.

Selection of one or more network axis can provide unique perspectives on glycoproteoform subnetworks. Axes selected by physiochemical properties (e.g., Mass, isoelectric point, hydrophobicity, etc.), monosaccharide compositions (F, H, GN, S), differences/ratios in monosaccharide content (e.g., GN-H, GN/H, etc.), and the like may be used. By selecting the network axes two or more distinct networks may be identified. This in turn may be used to discriminate between different glycans.

Intact mass spectral data of APPs and other glycoprotein markers can be used to generate a gp network based on pattern recognition of specific glycan mass shifts (e.g. Fuc, Hex, HexNAc, Neu5Ac, LacNAc, etc.) or simply linked via accurate composition assignments. Glycoproteoforms will be represented by nodes (denoted as a numerical set based on number of fucose, hexose, N-acetyl hexosamine, sialic acid, ie. F:H:GN:S) in the network and specific transferase-dependent mass shifts form the edges. Once generated, these networks are then arranged to visualize the transitive pathway between the least to most enzymatically processed gps. Networks layouts are expected to be unique for each glycoprotein, with node coordinates optimized depending upon the network size or compositional heterogeneity.

In some embodiments, a subnetwork may be determined. Exemplary subnetworks may be associated with post-translational modifications, SNPs, allotypes, etc. can be identified by probing any unassigned mass spectrometry data in the original data. The offshoot networks can be detected by assessing each base glycoproteofrom for delta masses that correspond to a certain type of PTM. The confidence that the PTM is real increases with the completeness of the off-shoot networks.

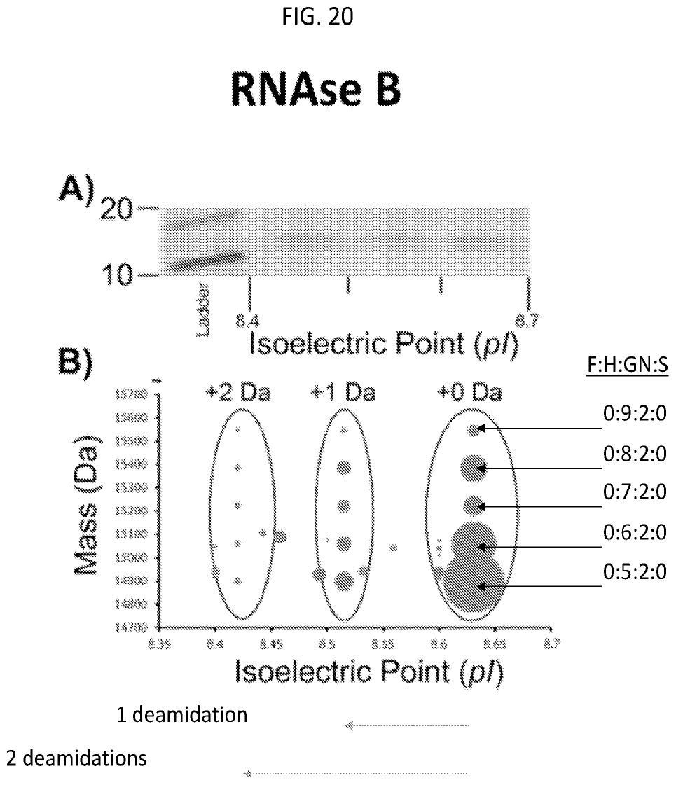

By way of example, pI separations to help differentiate deamidated forms of 5 glycoproteoforms of RNAseB. Off-shoot subnetworks may be assigned on the pI axis with either 1 or 2 deamidation events. +1 Am or +2 deamidation events may be associated with a 1 Da and 2 Da increase in mass relative to the base forms (respectively). (FIG. 20) Differences in mass, pI, or other physiochemical properties can be used to capture subnetworks associated with various forms of biological heterogeneity (e.g., PTMs such as phosphorylation, acetylation, etc., as well as, things like different allotypes/isoforms of the protein).

A centrality directed filtering approach may be used. Various centrality indices are used to assign a nodes importance in a static network given its position (e.g., eigenvector), shortest path relationships (e.g., betweenness), or number of connections to other nodes (e.g., degree). Similarly they may help optimize the network's connectivity through noise reduction algorithms. A node's closeness centrality value (c) measures it's distance to all other nodes, calculated using the inverse sum of the distance d(j,i) between the node of interest and all other reachable nodes. In some embodiments, c can be used to optimize a network's connectivity by elimination of poorly connected nodes (e.g., miss assigned gps due to spectral noise). A baseline gp connectivity score (GCS) may be determined by the Pearson product-moment correlation coefficient from linear least squares fit of rank ordered c for each gp. Then the GCS may be optimized with an “in-network” cutoff criteria at the largest Δc between the ranked nodes which—for a glycoprotein subject to conventional N-glycan biosynthesis rules—results in a fully connected network where each gp precedes or derives from another in a manner consistent with sequential monosaccharide addition by various glycosyltransferases.

Often one will attempt to reconstruct glycoproteoform mass spectra from plausible glycan information. Here, Inventors are attempting the reverse. Here, intact gp data is used to predict a site-independent N-glycan compositional distribution based on all gp observed in the network using one of two in silico calculation methods. The first is a conservative prediction of N-glycans with the putative F:H:GN:S are given a weighted frequency proportional to the intensity of the gp from which they are derived. Here the candidate N-glycans for each gp (e.g. a paired set of glycans for a diglycosylated protein, a triplet set for a triglycosylated protein, etc.) is calculated by dividing values for each gp sugar compositional unit (gpᵢ) by the number of glycosylation sites (NSG) and determines their nearest integers (└S(i)┘, ┌S(i)┐), resulting in a F:H:GN:S value that reflects an individual N-glycan composition that would be observed. This process is repeated for every unique gp composition and the intensity of each predicted N-glycan is the weighted cumulative intensity of the parent gp from which they were derived. This method yields the most abundant N-glycans on average and performs better when all glycosites have roughly symmetrical N-glycan biosynthesis. The second predictive method still utilizes the baseline gp/NSG calculation but allows for N-glycan compositions adjacent the predicted N-glycans in the bioprocessing pathway to also receive a portion of the overall intensity. By using a +1 hexose and N-acetyl glucosamine “tolerance” factor, predicted adjacent N-glycans (F:H+1:GN:S or F:H:GN+1:S) are given an additional 25% of the intensity of the primary composition.

In one embodiment, N-glycan compositional information is deduced with additional compensation factors for adjacent N-glycans. For example, using a +1 hexose and N-acetyl glucosamine “tolerance” factor, predicted adjacent N-glycans (F:H+1:GN:S or F:H:GN+1:S) are given an additional 5-25% of the intensity of the primary composition. This secondary method provides more flexibility in the N-glycan predictions when more data is acquired. In both cases, in silico predicted N-glycans discovered will be assigned structural topologies (i.e., degree of branching, bisection status) by a method that seeks to maximize the connectivity of the predicted N-glycan compositions to one another based on a pathway map generated from established rules of glycosyltransferase activity.

Numerous alternate forms of glycosylation such as N-acetyllactosamine addition or O-glycosylation can occur in tandem with standard N-glycosylation. These will typically confound predictions at the N-glycan level but will still be observed in the network as they too have mass shifts similar to those of N-glycans (Fuc, Hex, HexNAc, Sia). Differentiation of these events can occur at the network level. Firstly, Inventors can identify the largest possible glycoproteoform (gp_{N-MAX}) that could exist due to standard N-glycan biosynthesis. Combinatorial extrapolation of terminating structures based on the number of glycosylation sites allows for identification of gp_{N-MAX}=NSG*┌F┐:┌H┐:┌GN┐:┌S┐. These datapoints can be highlighted in the network and converge upon a gp value that reflects a maximum composition that can be derived from strictly N-glycan biosynthesis. This node may be used for determining non-N-glycan species as any nodes that exceed the gp_{N-MAX} compositional value is likely to contain an atypical glycosylation feature. For example, the total number of LacNAc residues is determined using the shortest path calculation between each paired set of gp containing the same ratio of hexose to N-acetyl hexosamine in the network (e.g. exactly a difference 0:1:1:0). If a node exists in the network that intersects this paired set, this node is considered an impeding node and the node pair is not designated as a LacNAc. Conversely, the lack of a shortest path indicates a higher likelihood of the LacNAc moiety as opposed to standard structural branching. O-glycosylation events can be discovered the same manner by looking at the compositional difference between all nodes that exceed gp_{N-MAX} and the critical node. This method will allow for compositional characterization of the accessory O-glycosylation event and can inform upon O-glycan core characteristics.

The disclosed methods may be used to comprehensively map gps in blood or other biospecimens and PNA can uncover relationships between altered gp expression and enzymatic activity. Identification of diagnostic or prognostic biomarkers in the biospecimen by the disclosed methods can be used to determine appropriate treatment for a subject.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

Visualization. Network graphs provide concise visualization of topological order or connectivity of complex data and facilitate determination of properties that describe or quantify the network, sub-structures, or individual nodes/edges. Networks and associated properties were generated to assess transitive pathway(s) between the least to most enzymatically processed gpᵢ observed in MS data with different topological arrangements applied to resolve gp subsets with shared oligosaccharide features (e.g., F, H, GN, S, LacNAc, etc). The network layouts are often unique for each G, with node (gpᵢ) coordinates that may be optimized depending upon the network size or compositional heterogeneity. Typically, the vertical axis is reflective of gp mass while the horizontal axis reflects dynamic separation based upon differences in sugar composition. For example, for gps identified for the di-glycosylated CSF L-PGDS different networks were generated to define gps clustered by sialic acid (network graphs, FIG. 5a-5b) or GlcNAc content (biographs, FIG. 5c), the two monosaccharides associated with glycan branch heterogeneity and termination, respectively. In network graphs, node size reflects MS intensity and data is arrayed vertically by increasing gp mass while the horizontal distribution is determined based on intensity-weighted importance of oligosaccharide content, e.g. F>S>GN. On the other hand, biographs were arrayed vertically by decreasing gp mass and separates along the X-axis by prominence of transferase activity (GN>SA) with gp intensity is associated with color hue.

Network Creation and Attributes. The networks connections are derived from gp assignments (F:H:GN:S). Each gpᵢ is iteratively given the possibility to connect to an adjacent node based on mass shifts of F, H, GN or S and will form an edge in the network only if the corresponding gpᵢ+F (or H, GN, S) also exists in the mass spectrum data. Each gpᵢ carries out the same iterative operation until all nodes and corresponding edges are determined. The formation of a biograph requires the creation of a sparse pairwise matrix where M is an N-by-N matrix where N is the total number of unique compositional gp in the mass spectra. M is a zeros matrix with logical values of 1 when two gp at their respective index, Mᵢⱼ, have a connecting node on the network.

Network Refinement and Glycoproteoforni Connectivity Score Optimization. Various centrality indices may be used to assign a nodes importance in a static network given its position (e.g., eigenvector), shortest path relationships (e.g., betweenness), or number of connections to other nodes (e.g., degree). Similarly they may help optimize the network's connectivity through noise reduction algorithms. A node's closeness centrality value (c) measures its distance to all other nodes, calculated using the inverse sum of the distance d(j,i) between the node of interest and all other reachable nodes. In our workflow, the c was used to optimize the gpᵢ∈G network's connectivity by elimination of poorly connected nodes (e.g., miss assigned gps due to spectral noise). A baseline network connectivity score, denoted Glycoproteoform Connectivity Score (GCS), was determined by the Pearson product-moment correlation coefficient from linear least squares fit of rank ordered c for each gp. The GCS was then optimized with an “in-network” cutoff criteria at the largest Δ c between the ranked nodes which—for a glycoprotein subject to conventional N-glycan biosynthesis rules—results in a fully connected network where each gp precedes or derives from another in a manner consistent with sequential monosaccharide addition by various glycosyltransferases. For example, in CSF L-PGDS the cutoff criterion resulted in a 0.14 improvement in GCS after elimination of 11 poorly connected nodes (FIG. 11). The summed intensities of the poorly connected gp was <3% of the total network signal with only 1 of the 11 removed from the network matching to predicted gp from previously reported bottom-up glycopeptide data. Manual inspection of the raw data suggests they were artifacts associated with poor deconvolution near the spectral baseline which highlights the potential utility of c indices to select against unlikely gpᵢ candidates.

Site-independent Prediction of “Standard” N-glycan Composition and Topology. F:H:GN:S compositions for each gpᵢ∈G network were used for site-independent predictions of probable standard N-glycan F:H:G:S compositions and intensity. The N-glycans predictions are typically constrained to ˜340 “standard” human N-glycans commonly observed in literature that range in complexity from high mannose to tetra-antennary with or without bisection of the central mannose by GlcNAc addition or core-GlcNAc fucosylation (FIG. 10). In each case, the predictions of the optimal structural topologies (i.e., degree of branching, bisection status) used for N-glycan connectivity visualization plots are deduced by a Predicted Pathway Optimization (PPO) method which assigns a Pathway Score (PS) that seeks to maximize the connectivity of the assigned N-glycan compositions to one another based on a pathway map generated from glycosyltransferase activity (FIG. 12).

Key Glycoproteoform Network and N-glycan Metadata. For all networks a set of gp-relevant metrics were also generated to quantify aggregated network or subnetwork attributes, including: (1) Intensity-weighted mean and intensity-weighted standard deviations for the F:H:GN:S or masses for the network or its sub-networks; (2) predicted number of glycosylation sites (NSG) (FIG. 13) and the most-probable bioprocessing pathway map for predicted N-glycans for the target protein (FIG. 12); (3) gp_{N-MAX} for all or part of a network which reflects that maximum predicted gp composition that can occur due to only N-glycan biosynthesis as dictated by (4)N-glycan_{MAX}—the terminating N-glycan for a N-glycan biosynthetic pathway tree (FIG. 2b), (5) the percentage of gp intensity that exceeds gp_{N-MAX}, and (6) any additional non-standard glycosylation features that were detected via the network tools (e.g. O-glycosylation compositions, LacNAcation events).

N-glycan Pathway and Databases. The biosynthetic pathway of N-glycans relies heavily upon a multitude of transferase activities, each with its own set of structural prerequisites. The logical pathway from combining these enzymatic rules makes it possible to generate a connected pathway of all N-glycans expressed within humans. Thmanuscript present Example uses 3 N-glycan databases derived from a subset of these pathways (FIG. 10): D1—75 unique compositions derived from the activity of 10 glycotransferase enzymes serves a generic and all-encompassing database⁶ that does not consist of peripheral fucosylation. D2—a modified form of D1 which contains both core and peripheral fucosylation in complex N-glycan structures but is also restricted to bi-antennary N-glycan structures that are most common to CSF-exclusive proteins. D3—a database curated specifically for α-1-antichymotrypsin analysis containing highly fucosylated N-glycans associated with increased α1,2, α1,3- or α1,4-fucosyltranfserase activity but without any sialation.

Estimating Number of Glycosylation Sites. Glycoproteins often contain N-glycans with ˜1 F in the -[Hex₃GlcNAc₂]-Asn core which permits naive approximation of the number of N-glycosites (NSG) from the intensity-weighted number of F residues observed across the network. However, to account for alternate glycan types (e.g., without non-fucosylated structures, polyLacNAc branch extensions, or O-glycans), we predicted all probable number of glycosites and then select the NSG containing the greatest number of matches (FIG. 13). For the network or each subnetwork an intensity-weighted mean number of fucose (F₀) is calculated which is used to determine a lower bound number of sites equal to └F₀┘−1 and an upper bound equal to ┌F₀┐+1, where the floor and ceil functions are used to calculate F₀ to the nearest integer. For Gₛₑₜ=[└F₀┘−1, └F₀┘, ┌F₀┐, ┌F₀┐+1] a list of gps is generated by binomial expansion

$$( \begin{matrix}n \\ k\end{matrix} ) ,$$

where k is the number of glycosylation sites and n is the number of N-glycans present in a curated database (D1, D2, or D3). The NSG is then determined from the maximum number of matches between the experimental data the theoretical gp list for each element in Gₛₑₜ.

N-Glycan Inference from Intact Data. Intact gp data is used to predict a site-independent N-glycan compositional distribution based on all gp observed in the network using one of two in silico calculation methods. The first is a conservative prediction of N-glycans with the putative F:H:GN:S are given a weighted frequency proportional to the intensity of the gp from which they are derived. Here the candidate N-glycans for each gp (e.g. a paired set of glycans for a diglycosylated protein, a triplet set for a triglycosylated protein, etc.) is calculated by dividing values for each gp sugar compositional unit (gpᵢ) by the number of glycosylation sites (NSG) and determines their nearest integers (└S(i)┘, ┌S(i)┐), resulting in a F:H:GN:S value that reflects an individual N-glycan composition that would be observed. This process is repeated for every unique gp composition and the intensity of each predicted N-glycan is the weighted cumulative intensity of the parent gp from which they were derived. This method yields the most abundant N-glycans on average and performs better when all glycosites have roughly symmetrical N-glycan biosynthesis. The second predictive method still utilizes the baseline gp/NSG calculation but allows for N-glycan compositions adjacent the predicted N-glycans in the bioprocessing pathway to also receive a portion of the overall intensity. By using a ±1 hexose and N-acetyl glucosamine “tolerance” factor, predicted adjacent N-glycans (F:H+1:GN:S or F:H:GN+1:S) are given an additional 25% of the intensity of the primary composition.

Gp_{N-MAX} Determination. The bioprocessing pathway of N-glycans is terminated by the activity of sialyltransferase and effectively limits the total possible compositions that can be made from N-glycans alone. FIG. 10 shows the possible terminating N-glycan structures within our N-glycan database (sans LacNAc extensions). By combinatorial extrapolation of these terminating structures, we can derive the largest gpᵢ that can occur from N-glycans only. These datapoints can be highlighted in the network and converge upon a gp value that reflects a maximum N-glycan-only composition: Gp_{N-MAX}=NSG*┌F┐:┌H┐:┌GN┐:┌S┐, assuming that a G contains at least some amount of N-glycans which are fully processed. The gp_{N-MAX} values in the networks are used to determine a critical node for determining non-N-glycan species that are observed in the mass spectrum (see Results).

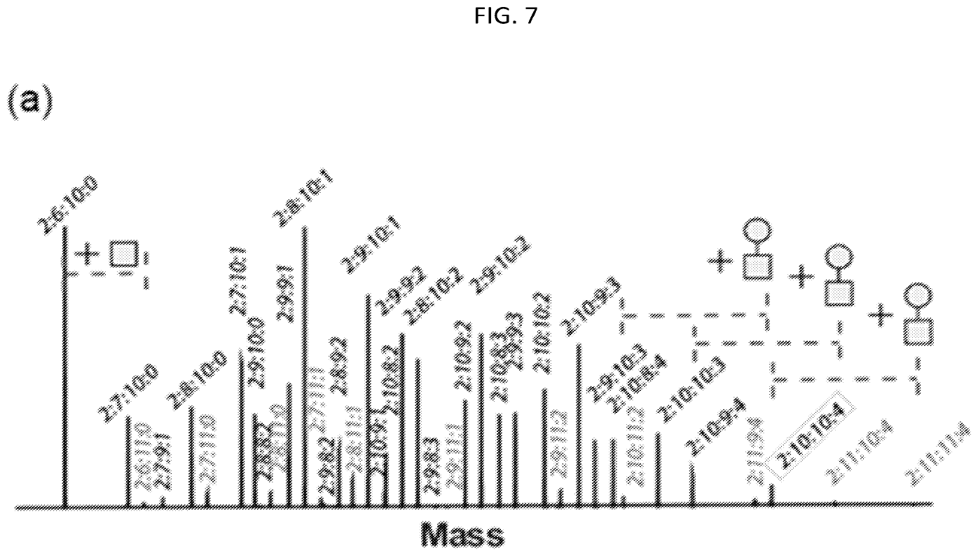

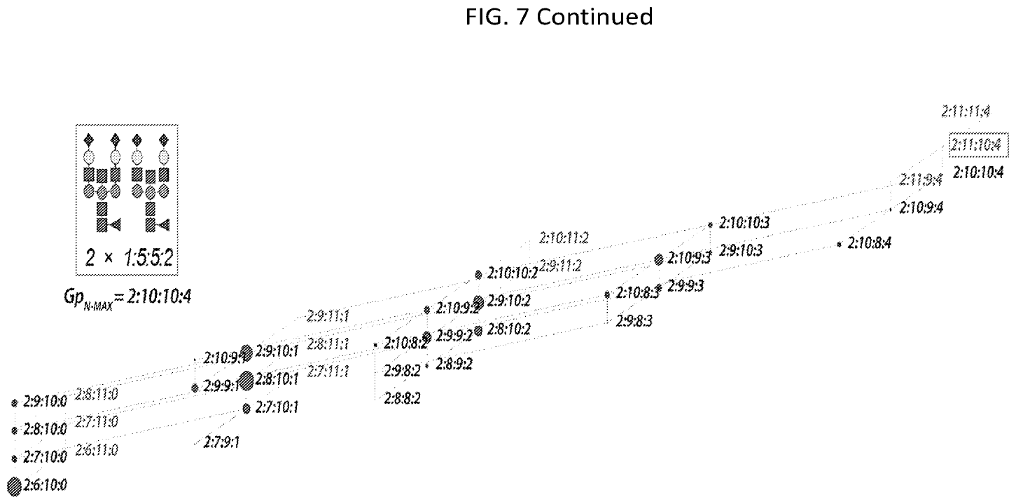

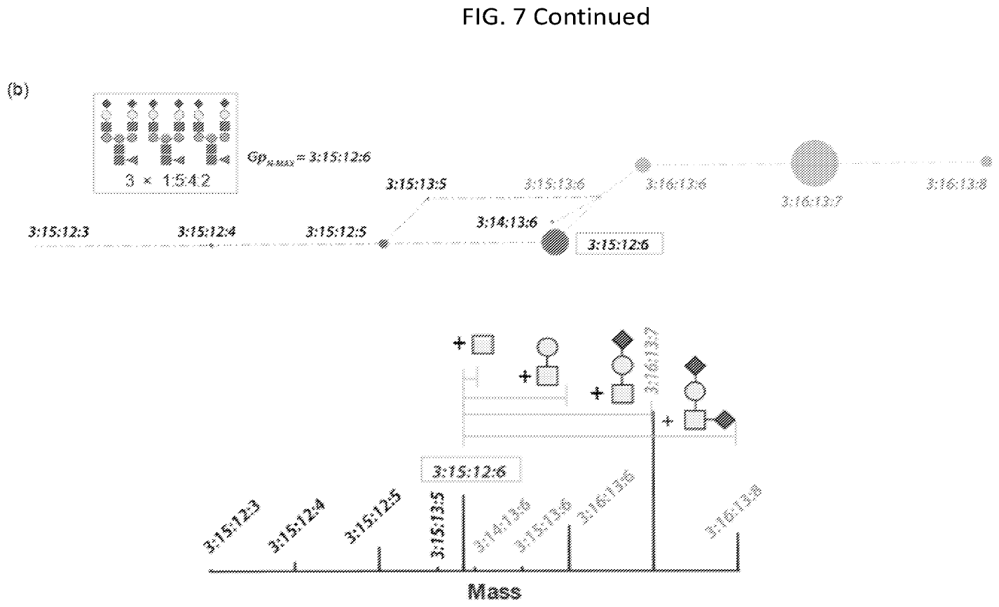

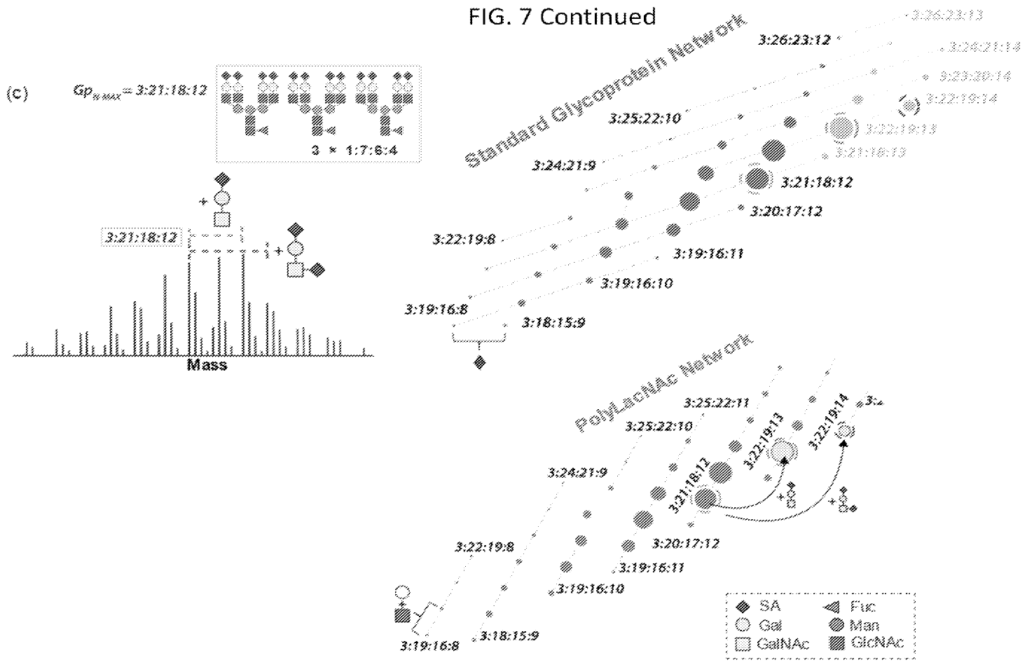

Estimating LacNAc Events. The total number of LacNAc residues is determined using the shortest path calculation between each paired set of gpᵢ in the standard network (FIG. 7c, blue) who have exactly a difference 0:1:1:0 (one H and one GN residue). If a node exists in the standard network that intersects this paired set, this node is considered an impeding node and the node pair is not designated as a LacNAc. For an adjacent node pair in the LacNAc network, if no shortest path exists between these two nodes in the standard network, then this pair is considered to contain the LacNAc moiety (FIG. 7c, red).

Bottom-Up Permutation. The theoretical construction of a top-down mass spectrum from bottom-up data utilizes a series of vectors where a vector's size is equivalent to the number of unique glycopeptide compositions. We generated multiple coordinate grids then concatenate the grid and then implement the reshape function (MATLAB) to a size equal to the number of glycosylation sites. This process generates a list of indices for each glycosylation site, which is then combined with intensity-weighted distributions for determining the theoretical top-down mass spectrum, either using products or summation to determine intensity values for every unique gp. Data comparison between any two mass spectra and/or N-glycan distributions uses the standard correlation coefficient.

Predicted Pathway Optimization. For a predicted set of N-glycan compositions determined in silico, each N-glycan is mapped onto the biosynthetic pathway (FIG. 10) via an optimization step in which all permutations within the set are scored based on connectivity:

$$Score = {\prod }_{j = 1}^{k} \frac{{n}_{j}}{{n}_{total}}$$

Where each set of N-glycans contains k of distinct unbroken sequential connections in the N-glycan pathway map. The score is the product value of the number of linked N-glycans in a set divided by the total number of N-glycan compositions (nₜₒₜₐₗ). FIG. 12 shows the score distribution (log scale) of all possible permutations of assignments for predicted L-PGDS N-glycans. The highest scoring pathway is then used as the most-probably pathway for the target glycoprotein. Notably, the scoring model shows significant separation and discrimination against more unlikely candidates. We use a normalized probability function to determine the likelihood that the optimal pathway is assigned due to chance:

$${p}_{i} = \frac{{f}_{i}}{N}$$

Where p is the probability of assignment, f is the frequency of a particular score and N is the total number of pathways assigned.

Software. The workflow described herein is executed in TDG-Vis.exe, a standalone application that was developed in MATLAB (MathWorks) using principles of graph theory to establish the gpᵢ∈G networks in both directional graphs and biographical views where nodes represent each gpᵢ and edges represent the shift in the sugar composition by a single monosaccharide (i.e., F, H, GN or S) or oligosaccharide element (e.g., LacNAc) (FIG. 2a). The current pipeline assesses a minimal dataset (user inputs require mass, intensity, and F:H:GN:S assignments, the latter of which can be performed by either commercially available tools or through accurate mass matching and has resources for manipulation, visualization, interpretation, and reporting on gp networks (see examples in Tables 2-6) (FIG. 2c).

### Intact Glycoprotein Analysis:

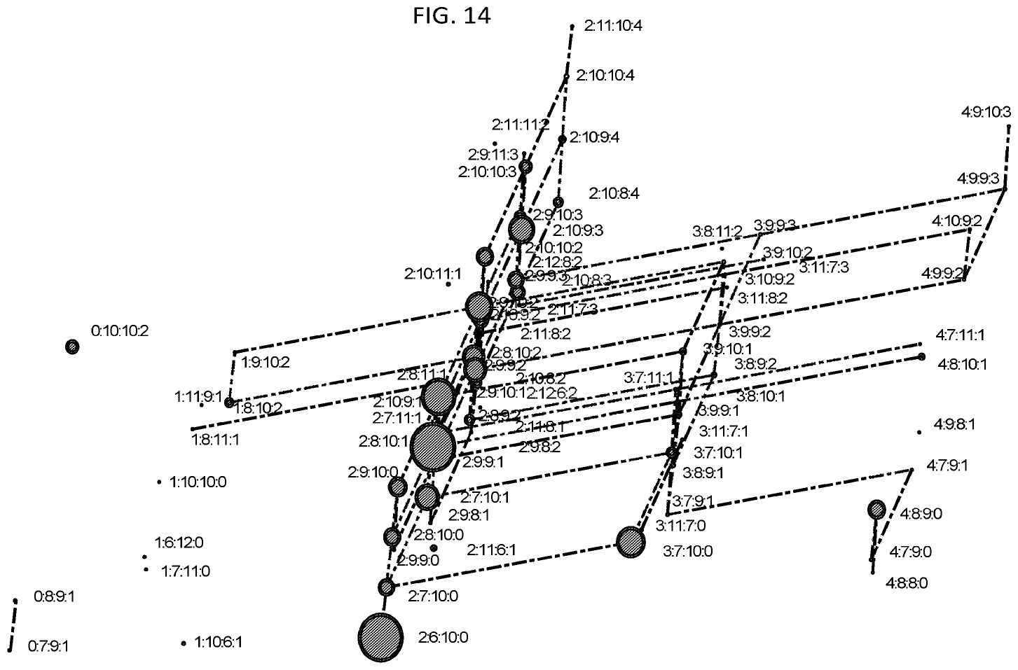

L-PGDS. CSF L-PGDS (or β-Trace) is an abundant glycoprotein in the central nervous system that is reported to consist predominantly of ‘brain-type’ N-glycans (i.e., bi-antennary N-glycans with the possibility of bisection) with minor abundances of O-glycosylation. The previously developed multi-dimensional top-down proteomics workflow enabled detection of differentially sialylated CSF L-PGDS gps derived from 15 N-glycans at two glycosylation sites (FIG. 6a). Application of PNA successfully echoed the previous findings, accurately identifying L-PGDS as a di-N-glycosylated protein (NSG=2) harboring ˜4086+409.05 Da of added oligosaccharides, but also provided potential etiological insights into L-PGDS gp heterogeneity. A GCS-optimized network showed 5 distinct subsets based on number of fucose (F₀₋₄∈L(P̌GDS) (FIG. 14) with a majority distributed between F₂ and F₃ (FIG. 5a), representing 81% and 12% of the total spectral intensity, respectively. Various calculated attributes for the dominant F₂∈LǦPGDS network included:

$$F : H : GN : S = [ 2 ] : [ 8.63 \pm 2.56 ] : [ 9.56 \pm 1.67 ] : [ 1.56 \pm 1.95 ] ;$$

$$wMass = 22 \; 772.42 \pm 409.05 ; N S G = 2 ;$$

$$\begin{matrix}gp \\ N - MAX\end{matrix} = ( \begin{matrix}{GN}_{8} = 2 : 10 : 8 : 4 \\ {GN}_{9} = 2 : 10 : 9 : 4 \\ {GN}_{10} = 2 : 10 : 10 : 4\end{matrix} ) ;$$

$$\begin{matrix}N - glycan \\ MAX\end{matrix} = ( \begin{matrix}1 : 5 : 4 : 2 , bi , non bisect \\ 1 : 5 : 5 : 2 , bi , bisect\end{matrix} ) ( \begin{matrix}1 : 5 : 4 : 2 , bi , non bisect \\ 1 : 5 : 5 : 2 , bi , bisect\end{matrix} ) ;$$

$$\begin{matrix}\% Int > \\ gp N - MAX\end{matrix} = 2.2 \% ; \begin{matrix}Other \\ ( O - glycan )\end{matrix} = ( \begin{matrix}GN , \\ H : GN\end{matrix} ) .$$

Inventors found F₂ could be divided into either Sₛ subsets (s=0, 1, . . . 4) with 6-11 gps per group (FIG. 5b) or into GN_{g} subsets (g=8-11) with 1-14 gps per group (FIG. 5c). The latter showed three “start compositions” (2:10:8:1, 2:8:9:1 and 2:6:10:0) associated with GN₈₋₁₀, respectively, which represent the least-enzymatically processed gps for the respective subnetworks. The directional arrangement of the network edges clearly showed stepwise addition of monosaccharides (+H, +GN, or +S) that is consistent with standard N-glycan biosynthesis that are sequentially regulated by GalT, GnT, and SiaT. In each case, the elaboration of each GN subnetwork converged toward a few maximally processed “terminal compositions”, gps=2:x:x:4 (where x=10 or 11). A quantitative dissection of key gp characteristics was shown in heatmaps of the summed intensity of the respective monosaccharides (FIG. 5b, bottom), as well as, a plot of the weighted H for S₀₋₄ (FIG. 5b, inset) which showed a positive correlation between H and S, reminiscent of the addition of a single S in an α2,3 or α2,6 linkage to a terminal galactose. Conversely, GN inversely correlated to S, ranging from approximately 10 GN to 8.6 GN for S₀ to S₄, respectively, which suggested that increased GlcNAcation (bisection) reduced the extent of glycan biosynthesis for L-PGDS. The proposed pathways and corresponding structures are shown in FIG. 5d.

Thirty-two putative N-glycans of varied abundance (FIG. 6b) were predicted by in-silico, site-independent, predictions from the L-PGDS network data with 14 of the 15 experimentally validated L-PGDS N-glycans (FIG. 6a) correctly predicted at frequencies that closely reflect the previously observed spectral intensities (r=0.75) (FIG. 6b). The PPO biosynthesis map (FIG. 12c) showed significant overlap of both the predicted and experimentally validated N-glycans with the bisected and non-bisected bi-antennary N-glycans pathways (FIG. 6d). Our predictions also resulted in an accurate determination of unique compositional ratio (e.g., abundance of 1-fucose vs 2-fucose N-glycans (appr. 96% vs 4%, respectively)). Interestingly, in the opposite comparison—bottom-up to intact data—the reconstruction of the theoretical intact mass spectrum from experimental N-glycan abundances (via either product or summed combinatorial permutation) resulted in poor similarity (r=0.26, FIG. 6c) where only 33 of 82 theoretical intact masses were observed experimentally.

As noted above, N-glycans from the PPO suggests that the most abundant (>20% r.a.) fully sialated N-glycans are both non-bisected and bisected bi-antennary structures (1:5:4:2 and 1:5:5:2, respectively) and represent the two maximally processed N-glycans (N-glycan_{Max}) within their respective biosynthesis pathways. Combinations of these compositions for a di-glycosylated protein resulted in three gp_{N-Max} compositions (2:10:8:4, 2:10:9:4, and 2:10:10:4) that were readily observed in the respective GN₈₋₁₀ subnetworks (FIG. 5c). However, the presence of low abundant gps exceeding the gp_{N-Max} for the GN_{g} subnetworks (i.e., 2:11:9:4, 2:11:10:4, and 2:11:11:4), representing 2.2% of the total network intensity, as well as the presence of in silico predicted N-glycans (<2% r.a.) suggested presence of alternate glycans (tri- or tetra-antennary N-glycans) or glycosylation events (e.g., O-glycosylation). While tri- or tetra-antennary N-glycans cannot be excluded based only on intact MS data, the prevalence of bisected bi-antennary N-glycans combined with the lack of connections from gps to compositions that are suggestive of tri-/tetra-antennary glycans (e.g., 2:12:10:4 or 2:10:12:4), Inventors postulated that L-PGDS gps exceeding gp_{N-Max}, containing either 11 hexose and/or GlcNAcs, harbor either O-[GN] or O-[H+GN]—both of which are indicated in the network and mass spectrum (FIG. 7a). While confirmation of the O-glycosylated proteoforms was not achieved by direct MS/MS (not shown), prior bottom-up glycopeptide analysis verified the existence of O—[H+GN] at low stoichiometry at Ser-7. Subtraction of these putative O-glycan moieties prior to in silico predictions of the N-glycans subsequently improved the correlation between the predicted and experimental N-glycan distributions (r=0.86).

EPO. Human recombinant erythropoietin (rhEPO) is a broadly used tri-N-glycosylated protein biotherapeutic that boosts erythrocyte production in a variety of chronic conditions. The glycosylation status of rhEPO is well known to impact its stability and efficacy. PNA was used to assess the biosimilarity of nMS datasets previously obtained for recombinant WT EPO and 4 different glycosyltransferase cell lines expressed in Chinese hamster ovary (CHO) cells intended to modify S content or the degree of N-glycan branching (Table 2). Comparison of the WT and cell line network attributes:

$$F : H : GN : S ( \begin{matrix}WT \\ SiaKO ( 98 ) \\ bi - ( 236 ) \\ tri - ( 112 ) \\ tetra - ( 113 )\end{matrix} ) = [ \begin{matrix}3 \\ 3 \\ 3 \\ 3 \\ 3\end{matrix} ] : [ \begin{matrix}22.19 \pm 1.47 \\ 22.66 \pm 2.65 \\ 15.66 \pm 0.49 \\ 22.2 \pm 2.04 \\ 21.79 \pm 1.58\end{matrix} ] : [ \begin{matrix}19.7 \pm 2.06 \\ 19.66 \pm 2.64 \\ 12.7 \pm 0.46 \\ 19.53 \pm 2.01 \\ 18.82 \pm 1.57\end{matrix} ] : [ \begin{matrix}12.63 \pm 1.45 \\ 1.56 \pm 0.87 \\ 6.51 \pm 0.89 \\ 9.75 \pm 1.64 \\ 11.43 \pm 1.52\end{matrix} ] ;$$

$$wMaxx = ( \begin{matrix}30003.24 \pm 994.42 \\ 29410.79 \pm 1079.98 \\ 28330.51 \pm 421.97 \\ 31758.93 \pm 950.94 \\ 32015.19 \pm 872.61\end{matrix} ) ; NSG = ( \begin{matrix}3 \\ 3 \\ 3 \\ 3 \\ 3\end{matrix} ) ; \begin{matrix}\% Int > \\ gp N - MAX\end{matrix} = ( \begin{matrix}76 \\ 77 \\ 69 \\ 62 \\ 60\end{matrix} )$$

$$\begin{matrix}gp \\ N - MAX\end{matrix} ( \begin{matrix}WT \\ SiaKO ( 98 ) \\ bi - ( 236 ) \\ WT Like tri - ( 112 ) \\ WT Like tetra - ( 113 )\end{matrix} ) = ( \begin{matrix}3 : 21 : 18 : 12 \\ 3 : 21 : 18 : 0 \\ 3 : 15 : 12 : 6 \\ 3 : 21 : 18 : 9 \\ 3 : 21 : 18 : 12\end{matrix} ) ;$$

$$\begin{matrix}N - glycan \\ MAX\end{matrix} = ( \begin{matrix}1 : \frac{6}{7} : \frac{5}{6} : \frac{3}{4} , \frac{tri}{tetra} , non bisect \\ 1 : 7 : 6 : 0 , tetra , non bisect \\ 1 : 5 : 4 : 2 , bi , non bisect \\ 1 : 7 : \frac{6}{7} : \frac{3}{4} , \frac{tri}{tetra} , \frac{non bisect}{bisect} \\ 1 : 7 : 6 : 4 , tetra , non bisect\end{matrix} ) ;$$

$$\begin{matrix}Other \\ ( Col 1 : LacNAc , \\ Col 2 , 3 : O - Glycan )\end{matrix} = [ \begin{matrix}H : GN \\ 2 \\ 8.5 \\ 1 \\ 4 \\ 4\end{matrix} ] [ \begin{matrix}H : GN : S \\ 1 \\ 1 \\ 1 \\ 1 \\ 1\end{matrix} ] [ \begin{matrix}H : GN : 2 S \\ 1 \\ 1 \\ 1 \\ 1 \\ 1\end{matrix} ]$$

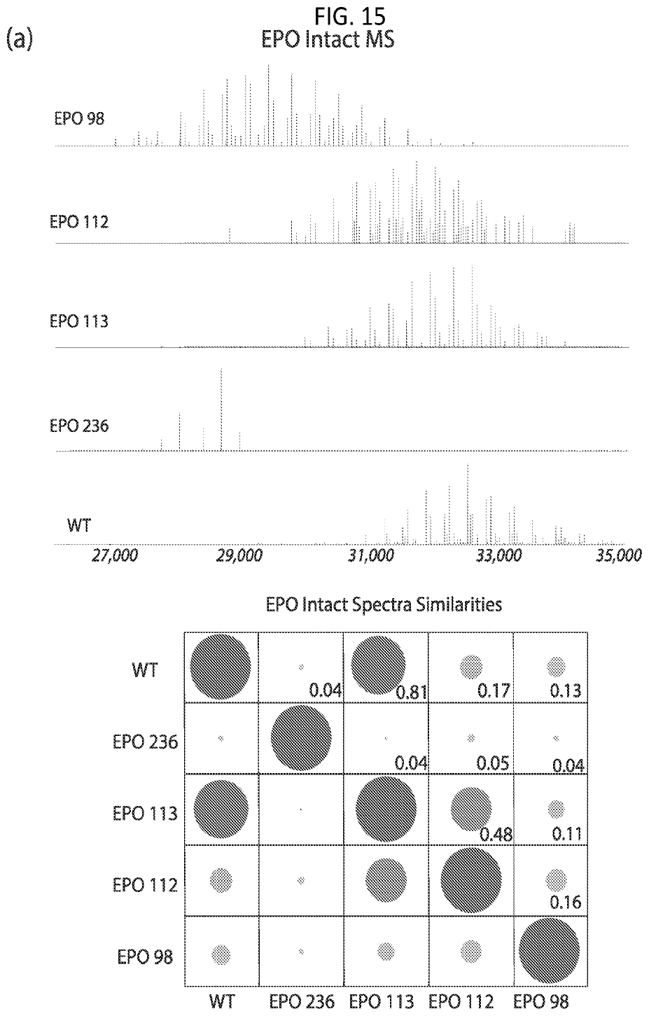

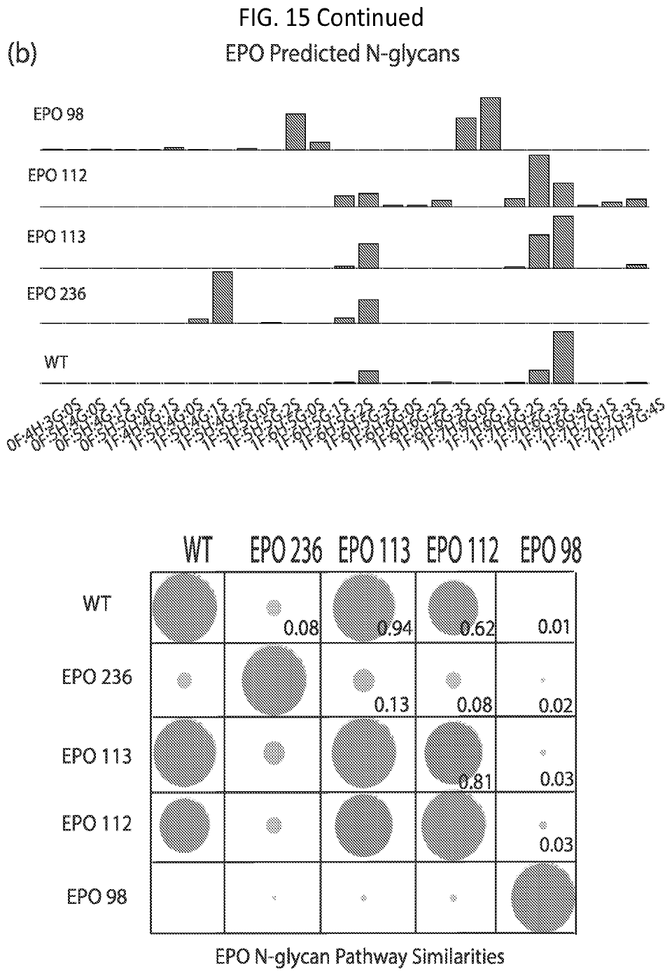

showed that despite relatively poor overlap of the spectral data (FIG. 15) the F, H, and GN content and gp_{N-Max} remained relatively consistent across different cell lines (except bi-antennary enriched cell line (EPO-236)). The N-glycan predictions and PPO approximations for each cell line predicted N-glycan structural characteristics (non-bisected, bi-, tri-, tetra-antenna) expected for each enrichment condition and were in agreement with previous middle-down characterization efforts associated with the nMS datasets. Weighted monosaccharide composition analysis suggested that dissimilarity between datasets was largely driven by S content, which was maximized for each level of branching enriched by KO conditions (i.e., 6, 9, and 12 for the bi-, tri- and tetra-antenna structures, respectively).

The PNA network attributes and plots also showed the EPO variants contained a significant number of gps with monosaccharide complexity exceeding the respective gp_{N-MAX} for each cell line, accounting for 60-77% of the total network signal which could be attributed to LacNAc addition in combination with potential O-glycosylation (e.g., FIG. 7b-c). Firstly, networks for most cell lines showed each was resolved into Sₛ subsets exhibiting stepwise addition of [H+GN]ᵢ between gps. For example, S₈₋₁₄ within EPO-113 contained up to 5 [H+GN] additions per subset (FIG. 7c) which were assigned as LacNAc additions due to a lack of impeding +H or +GN nodes via shortest path calculation in the primary network. Of note, the network attributes revealed the LacNAc addition within the sialyltransferase KO was highly favored in comparison with the other cell lines (i.e., addition of 8-9 vs 1-5 [H+GN], respectively) which was consistent with past work that shows S addition to branch structures serves to repress LacNAc addition.

Unexpectedly, PNA also provided a path to help elucidate confounding O-glycosylation events. For example, EPO-113 gps in S_{13,14} had S content that exceeded that allowable by the predicted gp_{N-MAX} (3:21:18:12). This was also noted for the WT EPO where the total weighted S content (S=12.63) exceeded that maximally allowed for fully sialylated tetra-antennary N-glycan (S=12). Similarly, EPO-98, 112, and 236 lines had S content exceeded that predicted by degree of N-glycan branching by ˜1 S. For EPO-113, inspection of gpᵢ intensity flow from gp_{N-MAX} (3:21:18:12) in S₁₂ to the adjacent S_{13,14} subsets showed that S_{12®13} network transition occurred by the addition of [H+GN+S] (i.e., 3:21:18:12 @3:22:19:13) while from S_{12®14} occurred by the addition of [H+GN+2S](i.e., 3:21:18:12 @3:22:19:14) (FIG. 7c). The results were consistent with the presence of Core 1 O-glycosylation to a significant subset of EPO gps and were consistent with O-glycans discovered in the WT EPO by middle-down characterization. Similar evidence for Core 1 O-glycans were obtained in the other cell lines where the progress of the O-glycan processing pathway could be traced by following the network path for compositions exceeding gp_{N-MAX}. For example, PNA on EPO-236, the cell line enriched for bi-antennary N-glycans, showed four gps with monosacharide complexity exceeding the predicted gp_{N-MAX}(3:15:12:6), consistent with contributions (appr. 20-25%) of Core 1 O-glycans HexNAc₁ and HexiHexNAc₁ (FIG. 7b).

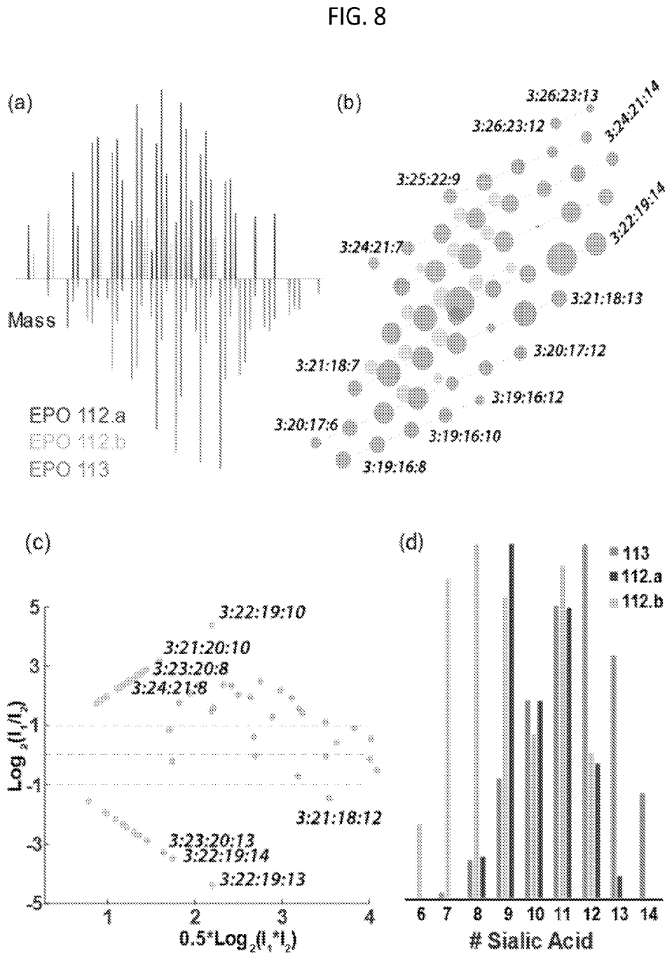

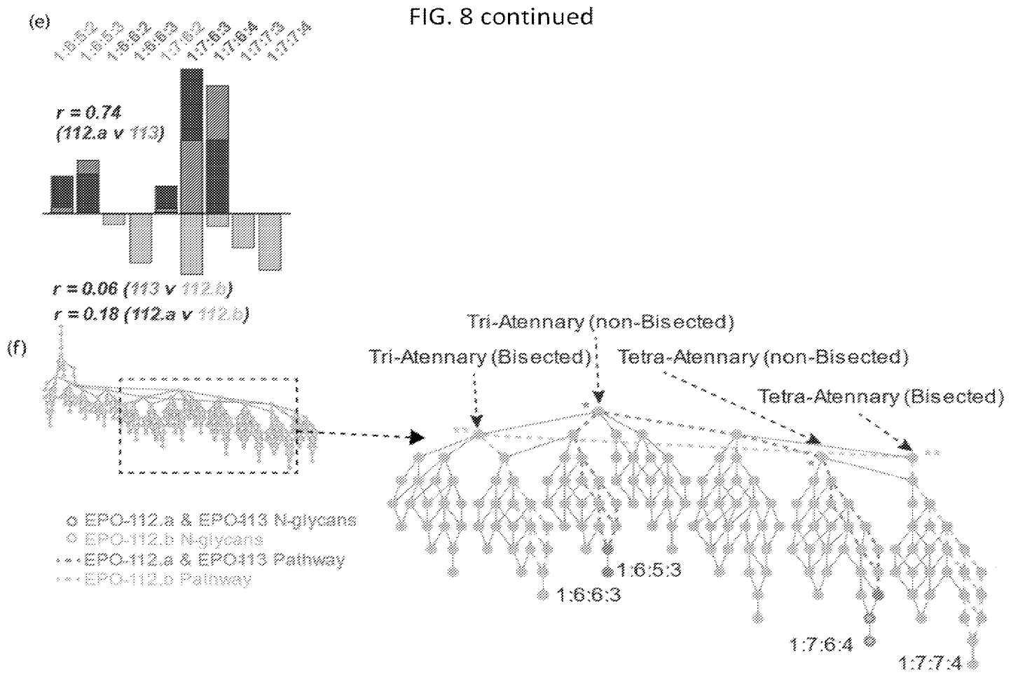

After accounting for LacNAcation and O-glycosylation, PNA N-glycan predictions from the intact gp network data provided unique and complementary insights into cell line biosimilarity (FIG. 15). For example, direct spectral correlation suggested that only EPO-113 had strong similarity to the WT (r=0.81), compared to other conditions (r_{avg}<0.17, FIG. 15a). However, the comparison of the predicted N-glycans for each cell line (FIG. 15b) showed significant improvements in similarity for both EPO-112 to WT lines (r=0.63 vs r=0.17) and EPO-113 to WT (r=0.94 vs r=0.81), as well as improved similarity between EPO-112 and EPO-113 (r=0.81 vs r=0.43). These improvements were attributed to the overlap in predicted N-glycan biosynthesis pathways which produce both tri- and tetra-antennary structures (1:6:x:3, 1:7:x:4, x=5 or 6) for all 3 datasets (FIG. 15b). Furthermore, visual comparison of EPO-112 and EPO-113 networks (FIG. 8) showed EPO-112 contained a unique gp subset (denoted EPO-112b) that accounted for ˜16% of the overall network intensity. At the predicted N-glycan level, poor correlation was observed between EPO-112b versus both EPO-112a (r=0.18) and EPO-113 (r=0.06) while EPO-112a versus EPO-113 showed a substantial improvement (r=0.74). The PPO analysis suggested that EPO-112a and EPO-113 consisted of non-bisected N-glycans (1:6:5:x or 1:7:6:x, x=2,3,4), while EPO-112b was expected to contain a mixture of non-bisected N-glycans along with a unique set of bisected tri- and tetra-antennary N-glycans (1:6:6:x or 1:7:7:x, x=2,3,4). Glycan biosynthesis maps of EPO variants (FIG. 8f) showed that EPO-112a and EPO-113 followed the strictly non-bisected N-glycan pathway while EPO-112b diverged early in the biosynthesis process, resulting in an additional series of bisected N-glycans that was responsible for much of the dissimilarity.

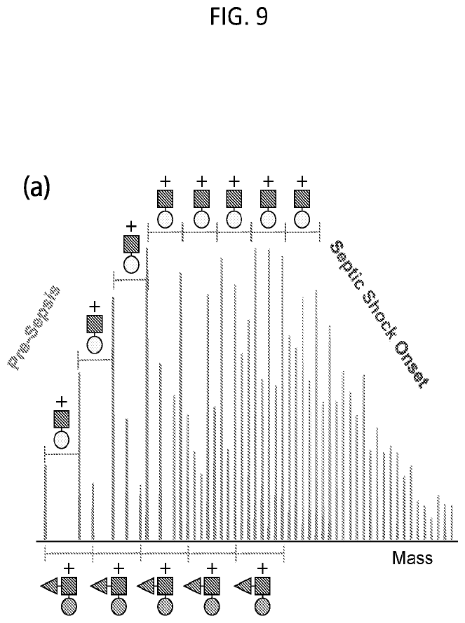

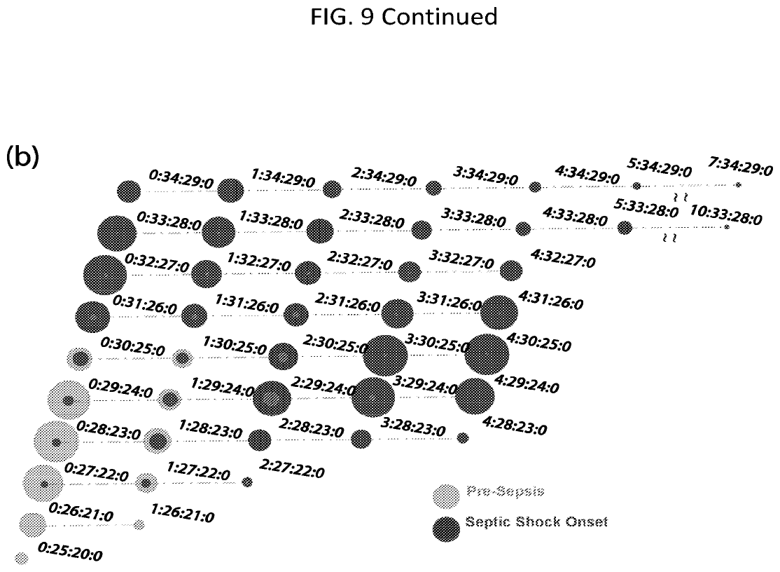

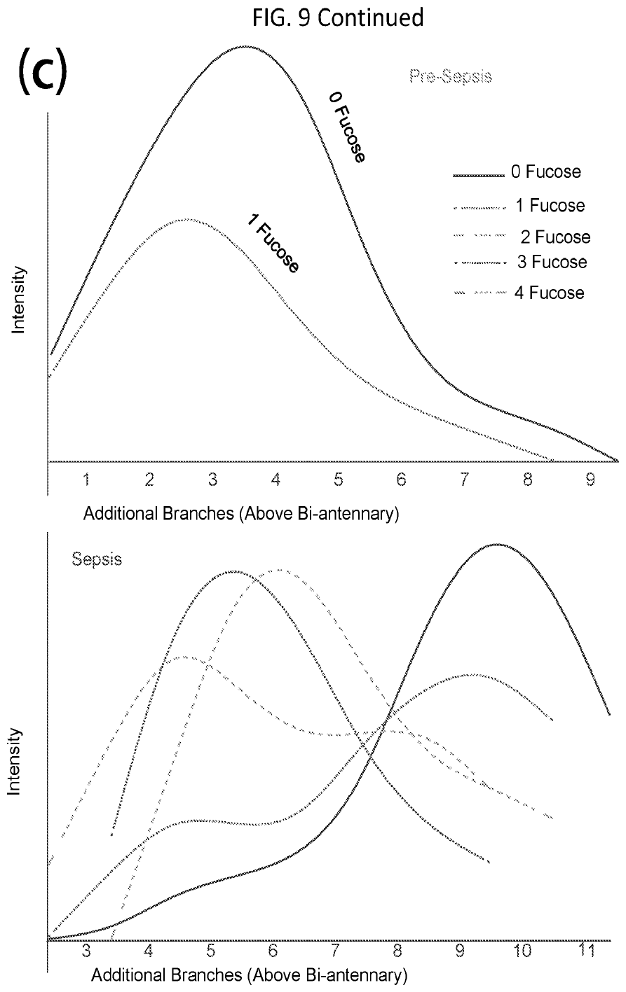

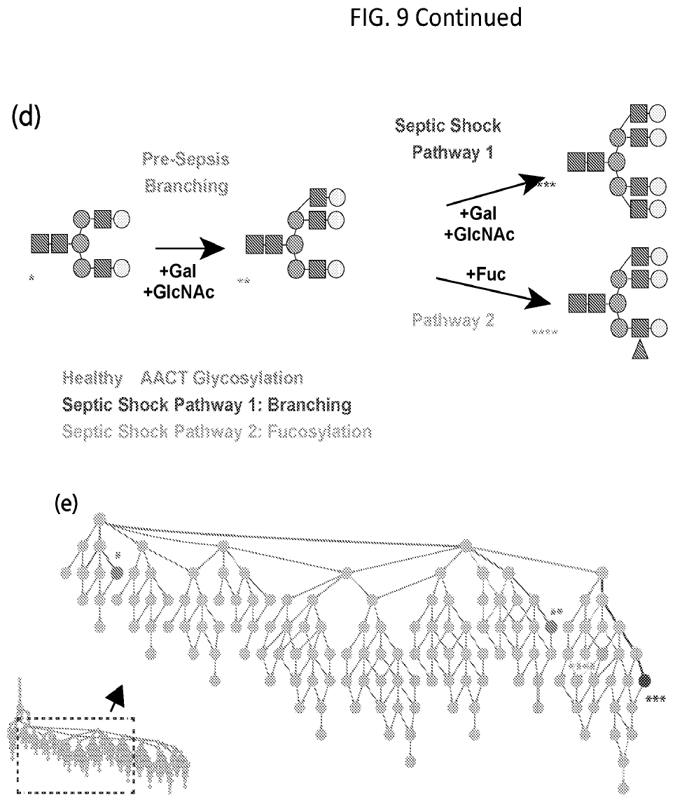

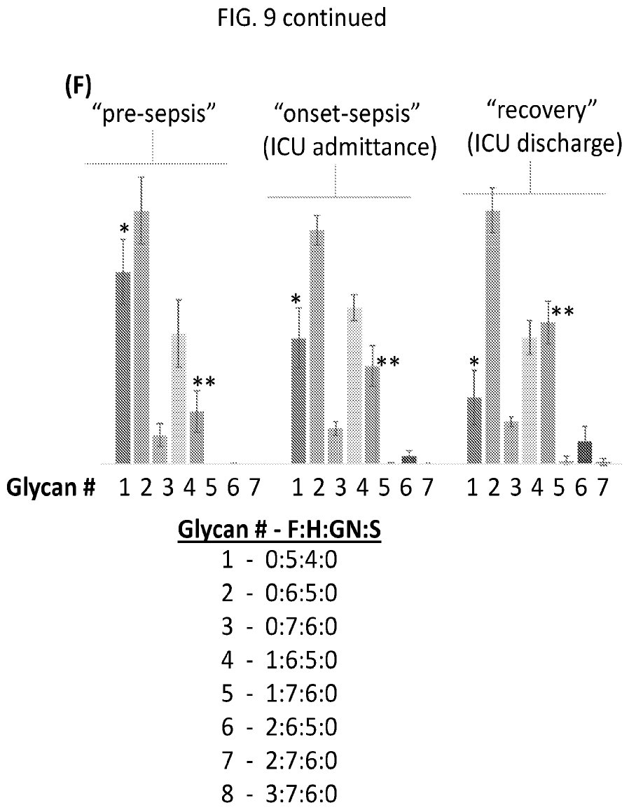

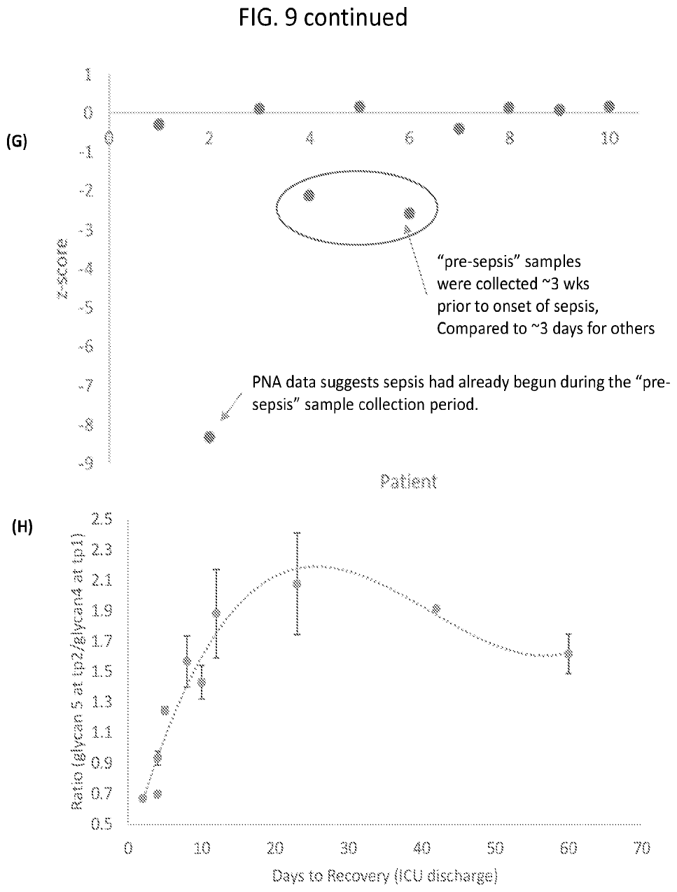

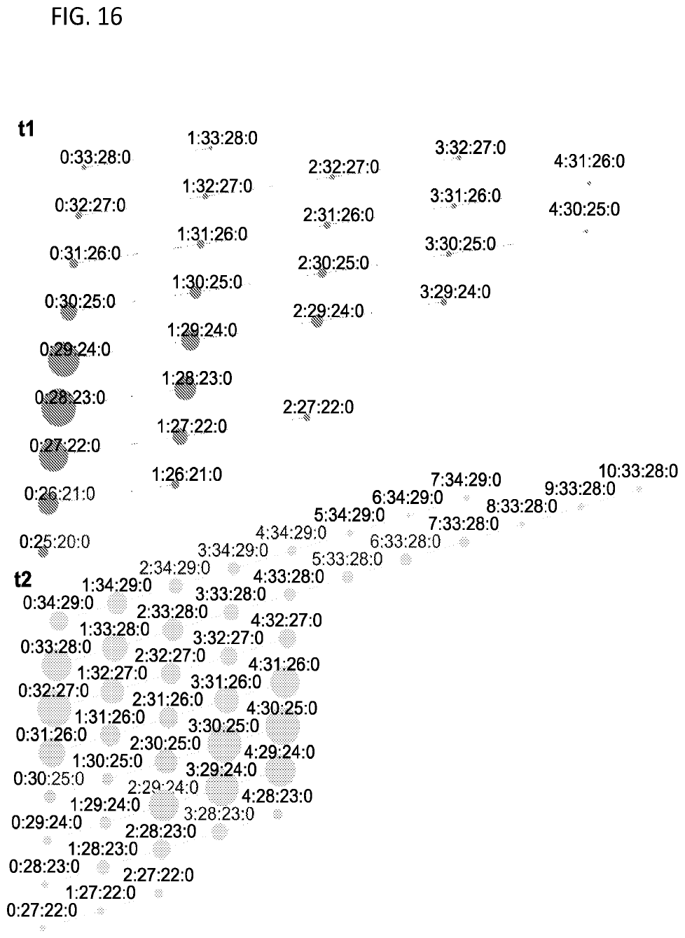

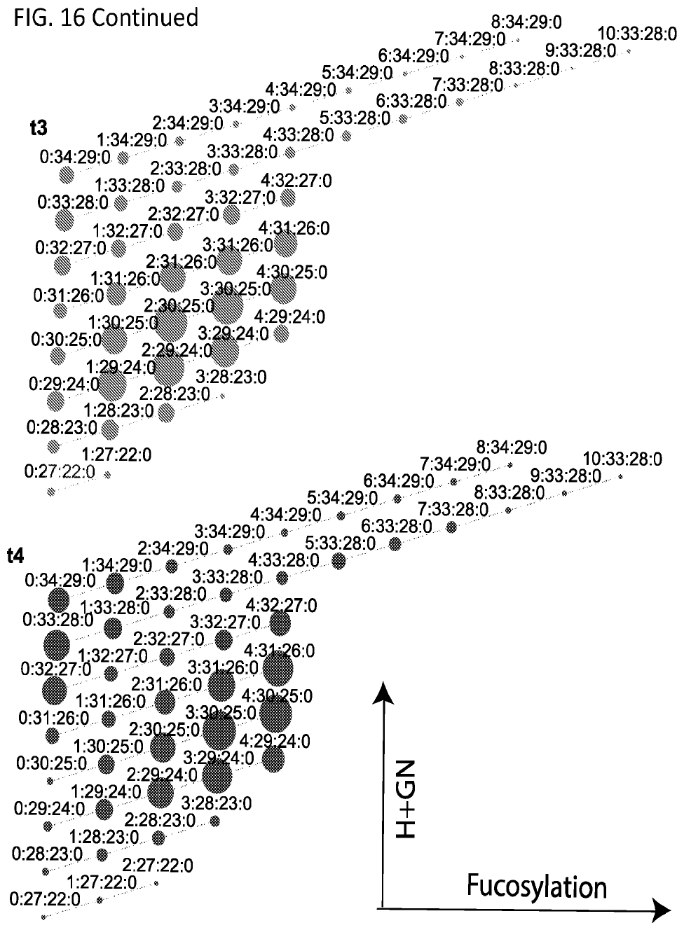

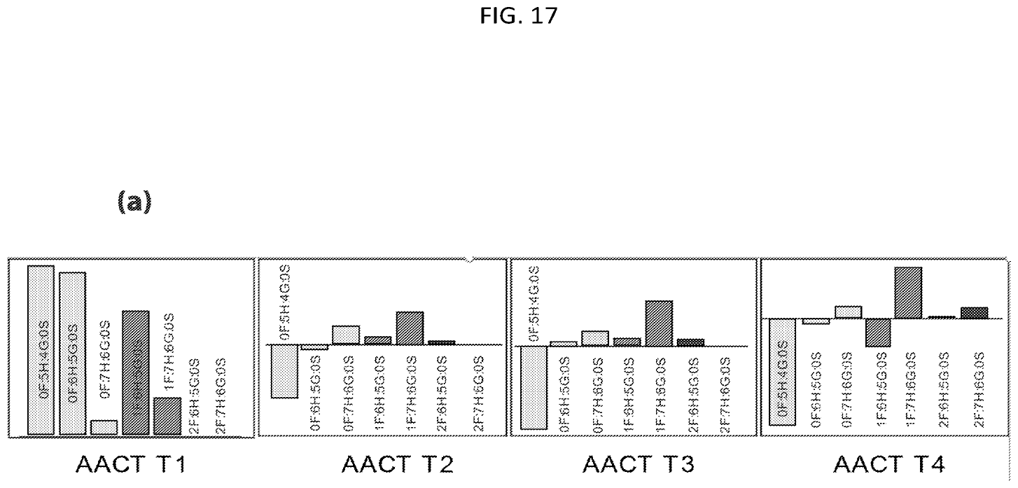

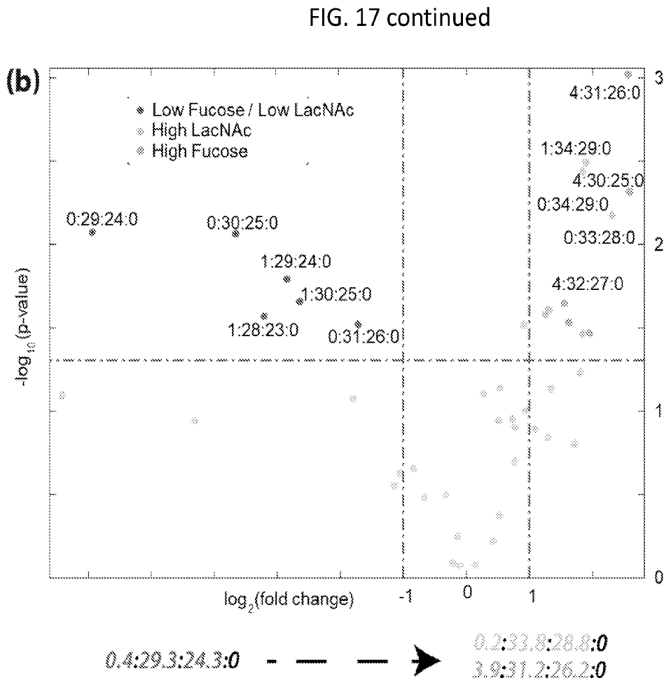

AACT. Alpha-1-antichymotrypsin (AACT) is an abundant plasma glycoprotein reportedly modified by largely bi- and tri-antennary N-glycans at up to five unique sites. Native MS investigations on de-sialated AACT gps from a small patient cohort diagnosed with septic shock found that enhanced N-glycan fucosylation (+F) and branching (+H and +GN) was observed at disease onset and remained throughout recovery. Inventors applied PNA to these AACT datasets to further elucidate glycoproteoform hierarchy and predicted N-glycan microheterogeneity at the different stages of acute septic shock (baseline (timepoint=t1), onset (t2), onset+1 day (t3), clinical resolution (t4)). Representative data for a single patient showed that each timepoint contained a complex stochastic distribution of gps arranged into F₀₋₁₀ subsets that exhibited a Δ[H+GN] arrangement (FIG. 9 and FIG. 16). While the Δ[H+GN]ᵢ topology is reminiscent of LacNAc addition observed in EPO, prior work suggested that AACT glycosylation profile in response to inflammation predominately results in enhanced branching from bi- to tri- to tetra-antenna structures. In the absence of less processed N-glycan intermediates these structures could result in a similar Δ[H+GN]ᵢ profile observed for LacNAc addition. Correlative analysis of t1-t4 exhibited moderate similarity between the pre-septic shock and those after onset (ₐᵥₑrₜ₁ᵥ₂₋₄=0.41) while comparisons of gps after onset of septic shock showed improved correlation trending in relation to the order of sample collection (rₜ₂ᵥ₃=0.97, rₜ₃ᵥ₄=0.89, rₜ₂ᵥ₄=0.81, respectively). The pre-sepsis (t1) network was dominated by F₀₋₁ subsets while sharp increases in F₂₋₄ gps occurred after the onset of septic shock (FIG. 9b and FIG. 16). The PPO results (FIG. 17a) for t1 suggested presence of primarily non-fucosylated, non-bisected di- and tri-antennary N-glycans (0:5:4:0 and 0:6:5:0, respectively) (FIG. 9b). The results indicated that >85% of the observed gps contained one or fewer predicted tri-antennary structures per glycosite, resulting in the observed pseudo-normal intensity distribution from 0:25:20:0 (all bi-antennary N-glycans) to 0:30:25:0 (all tri-antennary N-glycans) (FIG. 9b). After the onset of sepsis, a bimodal intensity distribution of gps (FIG. 9c) was observed, suggesting sepsis altered AACT glycosylation profiles with two divergent processing events. In the first, low fucosylated subsets (F₀₋₁) exhibited up to 9 Δ[H+GN]ᵢ additions with the original gp intensity profile pushed to between 0:30:25:0 to 0:34:29:0. This distribution is indicative of gps that are predicted to contain largely non-fucosylated, tri- and tetra antennary structures (FIG. 9d, upper path), or, lower branch structures elongated through LacNAc addition. In the second event, AACT was typically subject to only 3-6 [H+GN] additions along with 2-4 fucosylation events. Predictions of N-glycans for the second network intensity profile centered around 3:29:24:0 to 4:31:26:0 reflected enhanced expression of mono-fucosylated tri- or tetra-antennary structures (i.e., 1:6:5:0, 1:7:6:0) (FIG. 9d, lower path) which was in agreement with previous middle-down results. The lack of predicted 1:5:4:0 suggested that fucose incorporation occurs on peripheral branches versus at the N-glycan core. Fucosylation of various blood-based proteins have been proposed as biomarkers of various sepsis-subtypes, including α-1-acid glycoprotein (AGP) where differential regulation of bi-antennary structure versus those with increased fucosylation was predictive of survival patients. For AACT, the original investigation readily ascertained that changes in fucosylation and glycan branching occurs with the onset of septic shock; however, PNA further elucidated that AACT gps in response to sepsis may be subject to divergent substrate competition reactions that result in the observed bi-modal distribution (FIG. 9d). The suggested competition between branching and fucosylation pathways that is apparent in AACT gp networks has not been reported and may reflect diagnostic glycosylation profiles that would impact their function or longevity.

TABLE 2
Glycoproteins Characterized With PNA

| | | | | [H + GN]ᵢ | Analytical | | |
|---|---|---|---|---|---|---|---|
| Protein | Abbrv. | # N-Glycan | # O-Glycan | Series | Technique | Source | Ref |
| L-prostaglandin D-synthase (L- | L-PGDS | 2 | 1 | No | IEF-RPLC- | CSF - Human | 1 |
| PGDS) | | | | | FTMS | | |
| Wild-Type (WT) EPO | EPO | 3 | 1 | Yes | Native + MD | rh - CHO | 2 |
| | WsT | | | | MS | | |
| Sialyltransferase KO EPO | EPO 98 | 3 | 1 | Yes | EMR MS | rh - CHO | 3 |
| Tri-antennary EPO (WT-Like) | EPO 112 | 3 | 1 | Yes | EMR MS | rh - CHO | 3 |
| Tetra-antennary EPO (WT-Like) | EPO 113 | 3 | 1 | Yes | EMR MS | rh - CHO | 3 |
| Bi-antennary EPO | EPO 236 | 3 | 1 | Yes | EMR MS | rh - CHO | 3 |
| mAb Sample “A” | NIST | 1 | 0 | Yes | Cross Platform | NIST | 4 |
| | mAb A | | | | | | |
| α-1-antichymotrypsin (4 | AACT | 5 | 0 | Yes | Native TDMS | Plasma - | 5 |
| timepoints) | (t1-t4) | | | | | Human | |

recombinant human (rh); erythropeitin (EPO); monoclonal antibody (mAb); National Institue of Standards and Technology (NIST); Chinese Hamster Ovaries (CHO); cerebrospinal fluid (CSF)
1: Zhang, J. M.; Corbett, J. R.; Plymire, D. A.; Greenberg, B. M.; Patrie, S. M., Proteoform analysis of lipocalin-type prostaglandin D-synthase from human cerebrospinal fluid by isoelectric focusing and superficially porous liquid chromatography with Fourier transform mass spectrometry. Proteomics 2014, 14 (10), 1223-1231.
2: Yang, Y.; Liu, F.; Franc, V.; Halim, L. A.; Schellekens, H.; Heck, A. J. R., Hybrid mass spectrometry approaches in glycoprotein analysis and their usage in scoring biosimilarity. Nat Commun 2016, 7.
3: Caval, T.; Tian, W. H.; Yang, Z.; Clausen, H.; Heck, A. J. R., Direct quality control of glycoengineered erythropoietin variants. Nat Commun 2018, 9.
4: De Leoz, M. L. A. et al., NIST Interlaboratory Study on Glycosylation Analysis of Monoclonal Antibodies: Comparison of Results from Diverse Analytical Methods. Mol Cell Proteomics 2020, 19 (1), 11-30.
5: Caval, T.; Lin, Y. H.; Varkila, M.; Reiding, K. R.; Bonten, M. J. M.; Cremer, O. L.; Franc, V.; Heck, A. J. R., Glycoproteoform Profiles of Individual Patients' Plasma Alpha-1-Antichymotrypsin are Unique and Extensively Remodeled Following a Septic Episode. Front Immunol 2020, 11, 608466.

TABLE 3
Weighted average F:H:GN:S data for distinct gp subsets for L-PGDS. Bold entries
indicate fixed values while the remaining glycan-values are representative weighted
mean values with their respective standard deviations on the right-hand side.

| gp ∈ L - PGDS | | | | | | | | |
|---|---|---|---|---|---|---|---|---|
| (gp subset) | F | H | GN | S | F-Stdev | H-Stdev | GN-Stdev | S-Stdev |
| F₀ | 0 | 9.85 | 9.93 | 1.93 | 0 | 0.56 | 0.25 | 0.25 |
| F₁ | 1 | 8.32 | 9.49 | 1.74 | 0 | 0.86 | 1.44 | 0.53 |
| F₂ | 2 | 8.58 | 9.58 | 1.51 | 0 | 1.26 | 0.72 | 1.09 |
| F₃ | 3 | 7.60 | 9.76 | 0.62 | 0 | 0.99 | 0.63 | 0.75 |
| F₄ | 4 | 8.06 | 9.18 | 0.42 | 0 | 0.41 | 0.43 | 0.81 |
| H₆ | 2.00 | 6 | 10.01 | 0.00 | 0.06 | 0 | 0.11 | 0.00 |
| H₇ | 2.50 | 7 | 10.02 | 0.45 | 0.54 | 0 | 0.25 | 0.50 |
| H₈ | 2.30 | 8 | 9.78 | 1.06 | 0.81 | 0 | 0.46 | 0.75 |
| H₉ | 2.08 | 9 | 9.60 | 1.57 | 0.31 | 0 | 0.50 | 0.87 |
| H₁₀ | 1.81 | 10 | 9.08 | 2.60 | 0.59 | 0 | 0.84 | 0.76 |
| H₁₁ | 2.22 | 11 | 6.81 | 1.31 | 0.46 | 0 | 1.24 | 0.95 |
| H₁₂ | 2.00 | 12 | 6.66 | 2.00 | 0 | 0 | 0.94 | 0.00 |
| GN₆ | 1.77 | 10.83 | 6 | 1.06 | 0.42 | 0.52 | 0 | 0.24 |
| GN₇ | 2.75 | 11.00 | 7 | 1.39 | 0.43 | 0.00 | 0 | 1.35 |
| GN₈ | 2.03 | 9.99 | 8 | 2.88 | 0.22 | 0.29 | 0 | 0.82 |
| GN₉ | 2.31 | 9.10 | 9 | 1.92 | 0.71 | 0.75 | 0 | 1.04 |
| GN₁₀ | 2.06 | 8.06 | 10 | 1.05 | 0.55 | 1.21 | 0 | 0.94 |
| GN₁₁ | 2.19 | 7.97 | 11 | 1.09 | 0.51 | 1.17 | 0 | 0.42 |
| GN₁₂ | 1.00 | 6.00 | 12 | 0 | 0 | 0 | 0 | 0 |
| S₀ | 2.45 | 7.18 | 9.86 | 0 | 0.70 | 1.06 | 0.41 | 0 |
| S₁ | 2.16 | 8.33 | 9.73 | 1 | 0.48 | 0.93 | 0.81 | 0 |
| S₂ | 1.85 | 9.09 | 9.52 | 2 | 0.60 | 0.79 | 0.63 | 0 |
| S₃ | 2.05 | 9.66 | 9.09 | 3 | 0.29 | 0.49 | 0.70 | 0 |
| S₄ | 2.00 | 10.02 | 8.65 | 4 | 0.00 | 0.13 | 0.74 | 0 |

TABLE 4
Weighted average F:H:GN:S data for distinct gp subsets for EPO-112. Bold entries
indicate fixed values while the remaining glycan-values are representative weighted
mean values with their respective standard deviations on the right-hand side.

| gp ∈ EPO - 112 | | | | | | | | |
|---|---|---|---|---|---|---|---|---|
| (gp subset) | F | H | GN | S | F-Stdev | H-Stdev | GN-Stdev | S-Stdev |
| F₃ | 3 | 22.20 | 19.53 | 9.75 | 0 | 2.04 | 2.01 | 1.64 |
| H₁₈ | 3 | 18 | 15.00 | 9.00 | 0 | 0 | 0 | 0 |
| H₁₉ | 3 | 19 | 16.77 | 8.22 | 0 | 0 | 0.97 | 0.97 |
| H₂₀ | 3 | 20 | 17.58 | 8.68 | 0 | 0 | 0.91 | 2.22 |
| H₂₁ | 3 | 21 | 18.43 | 9.69 | 0 | 0 | 0.82 | 1.35 |
| H₂₂ | 3 | 22 | 19.26 | 10.11 | 0 | 0 | 0.67 | 1.23 |
| H₂₃ | 3 | 23 | 20.24 | 10.24 | 0 | 0 | 0.65 | 1.18 |
| H₂₄ | 3 | 24 | 21.12 | 10.05 | 0 | 0 | 0.47 | 1.57 |
| H₂₅ | 3 | 25 | 22.00 | 10.64 | 0 | 0 | 0.00 | 1.07 |
| H₂₆ | 3 | 26 | 23.40 | 10.32 | 0 | 0 | 0.80 | 1.90 |
| H₂₇ | 3 | 27 | 24.00 | 9.98 | 0 | 0 | 0.00 | 1.69 |
| H₂₈ | 3 | 28 | 25.00 | 11.00 | 0 | 0 | 0 | 0 |
| GN₁₅ | 3 | 18.00 | 15 | 9 | 0 | 0 | 0 | 0 |
| GN₁₆ | 3 | 19.00 | 16 | 7.55 | 0 | 0 | 0 | 0.50 |
| GN₁₇ | 3 | 20.00 | 17 | 8.92 | 0 | 0 | 0 | 1.33 |
| GN₁₈ | 3 | 20.80 | 18 | 9.51 | 0 | 0.60 | 0 | 1.34 |
| GN₁₉ | 3 | 21.57 | 19 | 9.67 | 0 | 0.82 | 0 | 2.14 |
| GN₂₀ | 3 | 22.49 | 20 | 10.22 | 0 | 0.87 | 0 | 1.17 |
| GN₂₁ | 3 | 23.57 | 21 | 10.07 | 0 | 0.82 | 0 | 1.48 |
| GN₂₂ | 3 | 24.51 | 22 | 10.60 | 0 | 0.86 | 0 | 0.96 |
| GN₂₃ | 3 | 25.70 | 23 | 9.92 | 0 | 0.71 | 0 | 1.76 |
| GN₂₄ | 3 | 27.00 | 24 | 9.98 | 0 | 0 | 0 | 1.69 |
| GN₂₅ | 3 | 27.12 | 25 | 11.44 | 0 | 0.99 | 0 | 0.50 |
| S₁ | 3 | 20 | 19 | 1 | 0 | 0 | 0 | 0 |
| S₆ | 3 | 20 | 17 | 6 | 0 | 0 | 0 | 0 |
| S₇ | 3 | 20.58 | 17.58 | 7 | 0 | 1.62 | 1.62 | 0 |
| S₈ | 3 | 21.95 | 19.07 | 8 | 0 | 2.36 | 2.31 | 0 |
| S₉ | 3 | 21.53 | 18.92 | 9 | 0 | 1.66 | 1.54 | 0 |
| S₁₀ | 3 | 22.31 | 19.77 | 10 | 0 | 1.90 | 1.85 | 0 |
| S₁₁ | 3 | 22.63 | 19.92 | 11 | 0 | 1.82 | 1.84 | 0 |
| S₁₂ | 3 | 23.13 | 20.48 | 12 | 0 | 2.06 | 2.13 | 0 |
| S₁₃ | 3 | 24.77 | 21.77 | 13 | 0 | 0.97 | 0.97 | 0 |

TABLE 5
Weighted average F:H:GN:S data for distinct gp subsets for EPO-113. Bold entries
indicate fixed values while the remaining glycan-values are representative weighted
mean values with their respective standard deviations on the right-hand side.

| gp ∈ EPO - 113 (gp subset) | F | H | GN | S | F-Stdev | H-Stdev | GN-Stdev | S-Stdev |
|---|---|---|---|---|---|---|---|---|
| F₃ | 3 | 21.79 | 18.82 | 11.43 | 0 | 1.58 | 1.57 | 1.52 |
| H₁₈ | 3 | 18 | 15 | 9 | 0 | 0 | 0 | 0 |
| H₁₉ | 3 | 19 | 16 | 9.50 | 0 | 0 | 0 | 0.89 |
| H₂₀ | 3 | 20 | 17 | 10.31 | 0 | 0 | 0 | 1.09 |
| H₂₁ | 3 | 21 | 18.10 | 10.93 | 0 | 0 | 0.30 | 1.29 |
| H₂₂ | 3 | 22 | 19 | 12.23 | 0 | 0 | 0 | 1.31 |
| H₂₃ | 3 | 23 | 20 | 12.04 | 0 | 0 | 0 | 1.32 |
| H₂₄ | 3 | 24 | 21 | 12.14 | 0 | 0 | 0 | 1.35 |
| H₂₅ | 3 | 25 | 22 | 11.93 | 0 | 0 | 0 | 0.91 |
| H₂₆ | 3 | 26 | 23 | 12.33 | 0 | 0 | 0 | 0.47 |
| GN₁₅ | 3 | 18 | 15 | 9 | 0 | 0 | 0 | 0 |
| GN₁₆ | 3 | 19 | 16 | 9.50 | 0 | 0 | 0 | 0.89 |
| GN₁₇ | 3 | 20 | 17 | 10.31 | 0 | 0 | 0 | 1.09 |
| GN₁₈ | 3 | 21 | 18 | 11.04 | 0 | 0 | 0 | 1.32 |
| GN₁₉ | 3 | 21.91 | 19 | 12.03 | 0 | 0.28 | 0 | 1.40 |
| GN₂₀ | 3 | 23 | 20 | 12.04 | 0 | 0 | 0 | 1.32 |
| GN₂₁ | 3 | 24 | 21 | 12.14 | 0 | 0 | 0 | 1.35 |
| GN₂₂ | 3 | 25 | 22 | 11.93 | 0 | 0 | 0 | 0.91 |
| GN₂₃ | 3 | 26 | 23 | 12.33 | 0 | 0 | 0 | 0.47 |
| S₇ | 3 | 21 | 18 | 7 | 0 | 0 | 0 | 0 |
| S₈ | 3 | 20.38 | 17.38 | 8 | 0 | 1.03 | 1.03 | 0 |
| S₉ | 3 | 20.31 | 17.31 | 9 | 0 | 1.58 | 1.58 | 0 |
| S₁₀ | 3 | 20.92 | 18.10 | 10 | 0 | 1.40 | 1.46 | 0 |
| S₁₁ | 3 | 21.56 | 18.56 | 11 | 0 | 1.39 | 1.39 | 0 |
| S₁₂ | 3 | 22.29 | 19.29 | 12 | 0 | 1.50 | 1.50 | 0 |
| S₁₃ | 3 | 22.65 | 19.65 | 13 | 0 | 1.23 | 1.23 | 0 |
| S₁₄ | 3 | 22.64 | 19.64 | 14 | 0 | 0.77 | 0.77 | 0 |

TABLE 6
Weighted average F:H:GN:S data for distinct gp subsets for α-1-antichymotrypsin
(AACT) at timepoint 1. Bold entries indicate fixed values while the
remaining glycan-values are representative weighted mean values with
their respective standard deviations on the right-hand side.

| gp ∈ AACT (t1) | | | | | | | | |
|---|---|---|---|---|---|---|---|---|
| (gp subset) | F | H | GN | S | F-Stdev | H-Stdev | GN-Stdev | S-Stdev |
| F₀ | 0 | 28.51 | 23.51 | 0 | 0 | 1.94 | 1.94 | 0 |
| F₁ | 1 | 29.08 | 24.08 | 0 | 0 | 1.81 | 1.81 | 0 |
| F₂ | 2 | 29.18 | 24.18 | 0 | 0 | 1.71 | 1.71 | 0 |
| F₃ | 3 | 29.57 | 24.57 | 0 | 0 | 1.67 | 1.67 | 0 |
| F₄ | 4 | 30.11 | 25.11 | 0 | 0 | 1.57 | 1.57 | 0 |
| F₅ | 5 | 33.71 | 28.71 | 0 | 0 | 0.46 | 0.46 | 0 |
| H₂₅ GN₂₀ | 0 | 25 | 20 | 0 | 0 | 0 | 0 | 0 |
| H₂₆ GN₂₁ | 0.21 | 26 | 21 | 0 | 0.40 | 0 | 0 | 0 |
| H₂₇ GN₂₂ | 0.95 | 27 | 22 | 0 | 1.04 | 0 | 0 | 0 |
| H₂₈ GN₂₃ | 1.19 | 28 | 23 | 0 | 1.18 | 0 | 0 | 0 |
| H₂₉ GN₂₄ | 1.44 | 29 | 24 | 0 | 1.29 | 0 | 0 | 0 |
| H₃₀ GN₂₅ | 1.71 | 30 | 25 | 0 | 1.31 | 0 | 0 | 0 |
| H₃₁ GN₂₆ | 1.84 | 31 | 26 | 0 | 1.39 | 0 | 0 | 0 |
| H₃₂ GN₂₇ | 1.67 | 32 | 27 | 0 | 1.39 | 0 | 0 | 0 |
| H₃₃ GN₂₈ | 1.31 | 33 | 28 | 0 | 1.29 | 0 | 0 | 0 |
| H₃₄ GN₂₉ | 1.92 | 34 | 29 | 0 | 1.53 | 0 | 0 | 0 |

1. A method for analyzing a glycoprotein (G), the method comprising:
identifying, with a processor from mass spectrometry data of the glycoprotein, a set of glycoproteoforms (gp∈G) where each of the glycoproteoforms (gpᵢ) have a measurable intact mass;
generating, with the processor from the identified set of glycoproteoforms (gp EG), a glycoproteoform network separated by saccharide features,
determining, with the processor from the glycoproteoform network, a site-independent prediction of N-glycans mapped to biosynthesis pathways; and
generating, with the processor from the determined N-glycans mapped to biosynthesis pathways, a glycan structure.

2. The method of claim 1, further comprising separating glycoproteoforms in a sample by a saccharide feature prior to obtaining the mass spectrometry data.

3. The method of claim 2, wherein the glycoproteoforms are separated by isoelectric focusing (IEF), capillary electrophoresis (CE), or hydrophilic interaction chromatography (HILIC).

4. The method of claim 3, wherein the glycoproteoforms are separated by saccharide content at different isoelectric points (pI).

5. The method of any one of claims 1-4, wherein gp sugar compositions are assigned by ensemble learning.

6. The method of claim 5, wherein gp sugar compositions are assigned by random-forest tree-bagger classification.

7. The method of any one of claims 1-6, wherein interconnected gp assignments are assigned by simulated annealing.

8. The method of any one of claims 1-7, wherein gp sugar compositions are assigned by mass alone.

9. The method of any one of claims 1-8, wherein a glycan topology is generated by selecting probable structures based on observed gp heterogeneity and eliminating erroneous assignments.

10. The method of claim 9, wherein erroneous compositional assignments are filtered out by centrality-discriminant scoring.

11. The method of any one of claims 1-10, wherein the mass spectrometry data has a spectral resolution of less than 2.2 Da.

12. The method of claim 11, wherein the mass spectrometry data has a spectral resolution between 1.0 and 2.0 Da.

13. The method of any one of claims 1-12 further comprising generating a network graph visualizing gp connectivity.

14. A method for analyzing a glycoprotein (G) in a subject, the method comprising:
obtaining a biospecimen from the subject;
analyzing, with a mass spectrometer, the biospecimen to generated mass spectrometry data of the glycoprotein;
identifying, with a processor from the mass spectrometry data of the glycoprotein, a set of glycoproteoforms (gp∈G) where each of the glycoproteoforms (gpᵢ) have a measurable intact mass;
generating, with the processor from the identified set of glycoproteoforms (gp∈G), a glycoproteoform network separated by saccharide features,
determining, with the processor from the glycoproteoform network, a site-independent prediction of N-glycans mapped to biosynthesis pathways; and
generating, with the processor from the determined N-glycans mapped to biosynthesis pathways, a glycan structure.

15. The method of claim 14 further comprising determining disease onset or disease recovery for the subject from the glycoproteoform network separated by saccharide features, the site-independent prediction of N-glycans mapped to biosynthesis pathways, the glycan topology, or any combination thereof.

16. The method of claim 15 further comprising administering a treatment to the subject in need of a treatment for disease onset.

17. The method of any one of claims 14-16, further comprising separating glycoproteoforms in a sample by a saccharide feature prior to obtaining the mass spectrometry data.

18. The method of claim 17, wherein the glycoproteoforms are separated by TEF, CE, or HILIC.

19. The method of claim 18, wherein the glycoproteoforms are separated by saccharide content at different isoelectric points (pI).

20. The method of any one of claims 14-19, wherein gp sugar compositions are assigned by ensemble learning.

21. The method of claim 20, wherein gp sugar compositions are assigned by random-forest tree-bagger classification.

22. The method of any one of claims 14-21, wherein interconnected gp assignments are assigned by simulated annealing.

23. The method of any one of claims 14-19, wherein gp sugar compositions are assigned by mass alone.

24. The method of any one of claims 14-23, wherein the glycan topology is generated by selecting probably structures based on observed gp heterogeneity and eliminating erroneous assignments.

25. The method of claim 24, wherein erroneous compositional assignments are filtered out by centrality-discriminant scoring.

26. The method of any one of claims 14-25, wherein the mass spectrometry data has a spectral resolution of less than 2.2 Da.

27. The method of claim 26, wherein the mass spectrometry data has a spectral resolution between 1.0 and 2.0 Da.

28. The method of any one of claims 14-27 further comprising generating a network graph visualizing gp connectivity.

29. A method for analyzing disease progression in a subject, the method comprising:
obtaining two or more biospecimens from the subject at different time points;
generating a glycoproteoform network or a glycan structure according to the methods according to any one of claims 14-28 for the two or more obtained biospecimens;
and identifying an indicia of disease onset or an indicia of disease recovery in the generated glycoproteoform network or the glycan structure between the two or more obtained biospecimens or a control.

30. A method for analyzing sepsis progression in a subject comprising the method according to claim 29, wherein the subject is diagnosed with sepsis or is suspected of developing, having, or having had sepsis.

31. The method of claim 30, wherein the glycoprotein is penta-N-glycosylated α-1-antichymotrypsin (AACT).

32. The method of claim 31, wherein the indicia of disease onset is a bimodal intensity distribution of gps, fucosylation, additional branching, or any combination there for penta-N-glycosylated α-1-antichymotrypsin (AACT).

33. The method of any one of claims 31-32, wherein the indicia of disease recovery is a ratio between 7:6 (H:GN) glycans at sepsis onset and 6:5 (H:GN) glycans pre-sepsis for penta-N-glycosylated α-1-antichymotrypsin (AACT).

34. A method for identifying disease onset or recovery in a subject, the method comprising:
generating a glycoproteoform network or a glycan structure for the subject according to the methods according to any one of claims 14-28; and
identifying an indicia of disease onset or an indicia of disease recovery in the generated glycoproteoform network or the glycan structure for the subject in comparison to one or more of pre-disease onset subjects, diseased subjects, or recovered subjects.

35. A method for analyzing sepsis onset or recovery in a subject comprising the method according to claim 34, wherein the subject is diagnosed with sepsis or is suspected of developing, having, or having had sepsis.

36. The method of claim 35, wherein the glycoprotein is penta-N-glycosylated α-1-antichymotrypsin (AACT).

37. The method of claim 36, wherein the indicia of disease onset is a bimodal intensity distribution of gps, fucosylation, additional branching, or any combination there for penta-N-glycosylated α-1-antichymotrypsin (AACT).

38. The method of any one of claims 34-37, wherein the indicia of disease recovery is a ratio between 7:6 (H:GN) glycans at sepsis onset and 6:5 (H:GN) glycans pre-sepsis for penta-N-glycosylated α-1-antichymotrypsin (AACT).
